(19) United States
(12) Patent Application Publication
(10) Pub. No.: US 2025/0276100 A1
(43) Pub. Date: Sep. 4, 2025

(54) DEVELOPMENT OF NTSR TARGETED AGENTS FOR IMAGING AND THERAPY APPLICATIONS

(71) Applicant: THE UNIVERSITY OF NORTH CAROLINA AT CHAPEL HILL, Chapel Hill, NC (US)

(72) Inventors: Zibo Li, Chapel Hill, NC (US); Zhanhong Wu, Chapel Hill, NC (US)

(21) Appl. No.: 18/862,823

(22) PCT Filed: May 3, 2023
(86) PCT No.: PCT/US2023/066537

§ 371 Date: Nov. 4, 2024

Related U.S. Application Data
(60) Provisional application No. 63/364,048, filed on May 3, 2022.

(51) Int. Cl. A61K 51/04

(52) U.S. Cl. CPC A61K 51/0453

(57) ABSTRACT

The invention relates to a new NTSR1 ligand, SR-CP-05, for targeted imaging and therapy. The invention also relates to a neurotensin receptor (NTSR1)-specific ligand that is suitable for radiolabeling and comprises a neurotensin ligand, a linker, and a chelator for binding a radioisotope. In some embodiments, a composition comprising the NTSR1-specific ligand is used for imaging, diagnosing, and/or guidance of treatment of a NTSR1-positive cancer.

### STATEMENT OF PRIORITY

This application claims the benefit, under 35 U.S.C. § 119(e), of U.S. Provisional Application No. 63/364,048, filed May 3, 2022, the entire contents of which is incorporated by reference herein in its entirety.

### STATEMENT REGARDING ELECTRONIC FILING OF A SEQUENCE LISTING

A Sequence Listing in XML format, entitled 5470-929WO_ST26.xml, 2,932 bytes in size, generated on May 3, 2023 and filed herewith, is hereby incorporated by reference into the specification for its disclosures.

### FIELD OF THE INVENTION

This invention relates to methods and compositions for imaging and therapy. In particular, the invention relates to neurotensin receptor (NTSR) targeted agents, their compositions, and methods for imaging and therapy using the same.

### BACKGROUND OF THE INVENTION

Prostate cancer is the most frequently diagnosed non-cutaneous malignancy and the second leading cause of cancer-related deaths among men in the US. Although various treatments have been developed, there is still an unmet need for greatly improved prostate cancer management. For example, gastrin-releasing-peptide-receptor (GRPR) targeted agents have demonstrated promising diagnosis and treatment potential. However, significant inverse correlations were found between GRPR expression and high prostate-specific antigen (PSA) values, greater tumor size, and higher Gleason scores in aggressive prostate cancer cases. This is problematic as it could lead to potentially false negative imaging results in particularly those tumors that should not be missed. In addition, current research results did not support GRPR as a target for late stage aggressive prostate cancer.

Prostate-specific membrane antigen (PSMA) expression was proven to be a prognostic factor for prostate cancer recurrence. Radiotracers based on monoclonal antibodies and other PSMA ligands have been developed for PSMA imaging, and encouraging results have been obtained in detecting prostate cancer early relapse after therapy. However, one major limitation with PSMA is its role in some advanced prostate cancers. Although androgen-dependent prostate cancer cell lines, including LNCaP, MDA PCa2b, and CWR22Rv1, express PSMA endogenously, it is also common that some advanced prostate cancer cells (e.g., androgen-independent PC3 or DU145) do not express PSMA. Significantly, for the PSMA positive LNCaP cell, knockdown of PSMA expression increased its invasiveness by 5-fold, suggesting PSMA could be down regulated as the tumor progresses.

While multiple factors could contribute to prostate cancer development, progression, and resistance to therapy, increasing evidence suggests that intra-prostate neuroendocrine-like cells play an important role in recurrent prostate cancers that become androgen independent. It is worth mentioning that prostate cancer could become enriched with (or entirely composed of) neuroendocrine cell clusters after long-term anti-androgen therapy. Secreted by neuroendocrine-like prostate cells, neurotensin (NTS) has numerous physiological effects predominantly mediated through its high affinity receptor NTSR1. Significantly, NTSR1 was found to be expressed and activated in aggressive prostate cancer cells, but not in normal prostate epithelial cells. In advanced prostate cancer, NTSR1 was recruited as an alternative growth pathway in the absence of androgens.

Additionally, claiming around 160,000 lives in the United States every year, lung cancer is the leading cause of cancer-related deaths in the United States. Lung cancer can be further categorized as non-small cell lung cancer (NSCLC) and small cell lung cancer (SCLC). Despite recent progress in lung cancer treatments, there is still an urgent unmet need to develop more effective therapeutic and prognostic methods for lung cancer management. NTS and its cognate receptor (NTSR1) are neuropeptide-receptor complexes frequently deregulated during the neoplastic process.

When NTS binds to NTSR1, phosphatidyl inositols are hydrolyzed leading to Ca²⁺ mobilization and protein kinase C (PKC) activation. NTSR1 activation leads to cell proliferation, survival, mobility, and invasiveness in specific cancer cell types. In early stage NSCLC, NTSR1 positivity has been found to associate with lower patient survival rates. NTS is one of the 73 genes overexpressed in the highly metastatic human lung cell line, H460-M, as compared to control cells. NTSR1 inhibitor was also found to impact mobility and proliferation of lung cancer cells NCI-H209 and H345, and inhibit the tumor growth of NCI-H209. High concentrations of NTS are also present in and secreted from half classic SCLC cells. Additionally, many other cancer types are positive for NTSR1, including pancreatic, colorectal, and breast cancer.

There is a need to develop new therapeutic, diagnostic, and prognostic methods for the management of NTSR1-positive cancers, which may complement existing approaches to achieve precision treatment.

### SUMMARY OF THE INVENTION

The present invention is based on the development of new NTSR1 ligands, such as SR-CP-05, for targeted imaging and therapy. The newly discovered SR-CP-05 ligand has high and persistent tumor uptake with only a minimal amount of tumor wash out after two days post injection.

One aspect of the invention is a neurotensin receptor (NTSR1)-specific ligand that is suitable for radiolabeling and comprises a neurotensin ligand, a linker, and a chelator for binding a radioisotope. In some embodiments, the radioisotope is selected from ⁶⁴Cu, ⁶⁷Cu, ⁶⁸Ga, ¹⁷⁷Lu, ¹⁸F, ⁹⁰Y, ²²⁵Ac, ²²⁷Th, ²²³Ra, ²¹³Bi, ²¹¹At, ²¹²Pb, ²¹²Bi, ²³⁰U, ²²⁶Th, and ¹⁴⁹Tb.

In some embodiments, the chelator is one or more of NOTA, DOTA, Cross Bridge-Cyclam, Cross Bridge-TE2A, DF chelators, DiAm-Sarcage, and derivatives thereof. In other embodiments, the linker is selected from NOTA, DOTA, Cross Bridge-Cyclam, Cross Bridge-TE2A, Sarcage, and derivatives thereof, polyamines, PEG, di-vinyl sulfones, di-halogenated alkyls, phenylene isothiocyanate, p-phenylene diisothiocyanate, 1,4-diazinane, phosphates, and combinations thereof.

In some embodiments, the NTSR1-specific ligand comprises formula I.

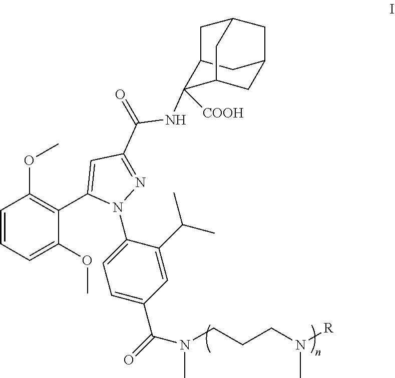

wherein n is 1-3; and R is a chelator.

In other embodiments, the NTSR1-specific ligand comprises formula VII:

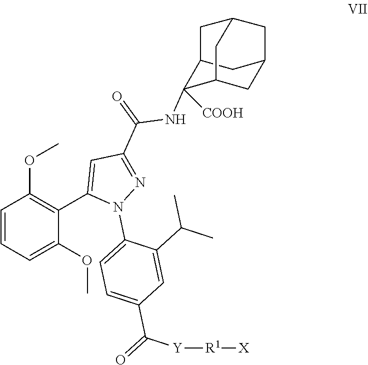

wherein Y is a bond or is selected from PEG, di-vinyl sulfone, di-halogenated alkyls, phenylene isothiocyanate, p-phenylene diisothiocyanate, 1,4-diazinane, and phosphates; X is H or LR²;
L is selected from PEG, di-vinyl sulfone, di-halogenated alkyls, phenylene isothiocyanate, p-phenylene diisothiocyanate, 1,4-diazinane, and phosphates; and
R¹ and R² are chelators.

Another aspect of the invention is a composition comprising the NTSR1-specific ligand. In some embodiments, the composition is used in the imaging, diagnosing, and/or guidance of treatment of a NTSR1-positive cancer.

One aspect of the invention relates to a method of carrying out a PET scan on a subject, comprising administering to the subject a ligand, probe, and/or composition of the present invention.

Another aspect of the invention provides a method of imaging tissue comprising a NTSR1-positive cancer in a subject, comprising administering to the subject a ligand, probe, and/or composition of the present invention.

Another aspect of the invention provides a method of imaging prostate cancer in a subject, comprising administering to the subject a ligand, probe, and/or composition of the present invention.

Another aspect of the invention provides a method of imaging lung cancer in a subject, comprising administering to the subject a ligand, probe, and/or composition of the present invention.

Another aspect of the invention provides a method of identifying NT SRI-positive cancer tissue in a subject, comprising carrying out a PET scan on the subject using a ligand, probe, or composition of the present invention, wherein the PET scan identifies the presence of NTSR1-positive cancer tissue.

Another aspect of the invention provides a method of treating NTSR1-positive cancer tissue in a subject, comprising administering the ligand, therapeutic agent, or composition of the present invention to the subject.

Another aspect of the invention provides a method of removing NTSR1-positive cancer tissue in a subject, comprising: carrying out a PET scan on the subject using a ligand, probe, and/or composition of the present invention, wherein the PET scan identifies the presence of NTSR1-positive cancer tissue; and surgically excising the identified NTSR1-positive cancer tissue, thereby removing the NTSR1-positive cancer tissue.

Another aspect of the invention provides a method of determining suitability of a subject with NTSR1-positive cancer or a subject at risk for or suspected to have or develop NTSR1-positive cancer for surgical removal of cancer tissue, comprising (a) carrying out a PET scan on the subject using a ligand, probe, and/or composition of the present invention, wherein the PET scan identifies the presence of NTSR1-positive cancer tissue; and (b) identifying the presence of NTSR1-positive cancer tissue, wherein the presence of NTSR1-positive cancer tissue indicates suitability of the subject for surgical removal of cancer tissue.

Another aspect of the invention provides a method of treating NTSR1-positive cancer in a subject, comprising predicting the suitability of a subject with NTSR1-positive cancer or a subject at risk for or suspected to have or develop NTSR1-positive cancer to surgical removal of cancer tissue by carrying out a PET scan on the subject using a ligand, probe, or composition of the present invention wherein the PET scan identifies the presence of NTSR1-positive cancer tissue, and treating the NTSR1-positive cancer based on the results of the PET scan.

Another aspect of the invention provides a method of treating a disorder of a NTSR1-positive tissue in a subject, comprising determining the suitability of a subject with the disorder or a subject at risk for or suspected to have or develop the disorder to treatment thereof by carrying out a PET scan on the subject using a ligand, probe, or composition of the present invention wherein the PET scan identifies the presence of NTSR1-positive tissue, and treating the disorder based on the results of the PET scan.

These and other aspects of the invention are set forth in more detail in the description of the invention below.

### BRIEF DESCRIPTION OF THE DRAWINGS

FIG. 1 shows companion and theranostic approaches in nuclear medicine. Molecular imaging uses a PET probe to detect NTSR positive lesion. Radionuclide based therapy will treat the NTSR1 positive tumor thereafter.

FIG. 2A shows NTSR1 is overexpressed on prostate cancer samples, but not normal prostate. FIG. 2B shows NTSR1, PSMA and GRPR staining on prostate cancer samples.

FIG. 3A shows microPET images of athymic male nude mice bearing PC3 tumor with ¹⁸F-VS-Cys-NTSₘᵤₜ. The arrow indicates tumor. FIG. 3B shows biodistribution of ¹⁸F-VS-Cys-NTSₘᵤₜ in PC-3 tumor-bearing nude mice.

FIG. 4 shows representative images for radiolabeled NTSR1 probes.

FIG. 5 shows a western blot analysis of NTSR1 expression in normal mouse organs and PC3 xenograft.

FIG. 6 shows a western blot demonstrating NTSR1 expression in human lung cancer cell lines.

FIG. 7 shows a western blot demonstrating NTSR1 expression in human prostate PC3, DU145 and C4-2B cells, but not in LNCaP cells.

FIG. 8 shows ⁶⁴Cu labeled SR-CP-05 outperforms both NT peptide analogs and other agents based on SR142948A including ⁶⁴Cu-3BP-227 in a side by side comparison.

FIG. 9 shows a general structure of SR-CP-05 based agents.

FIG. 10 shows a reaction scheme for forming one embodiment of SR-CP-05 based agents.

FIG. 11 shows the in vivo distribution pattern of a dual modality probe (DOTA-K(Cy5.5)-Ahx-DGEA) demonstrating a discrepancy between optical and PET imaging.

FIG. 12 shows the ex vivo distribution pattern of a dual modality probe (DOTA-K(Cy5.5)-Ahx-DGEA) demonstrating a discrepancy between optical and PET imaging (A kidney, B heart, C liver, D spleen, E lung, F tumor), which shows the need for developing stable ⁶⁴Cu chelators.

FIG. 13 shows tumor accumulation of SR-CP-18 at 24 hrs (high contrast).

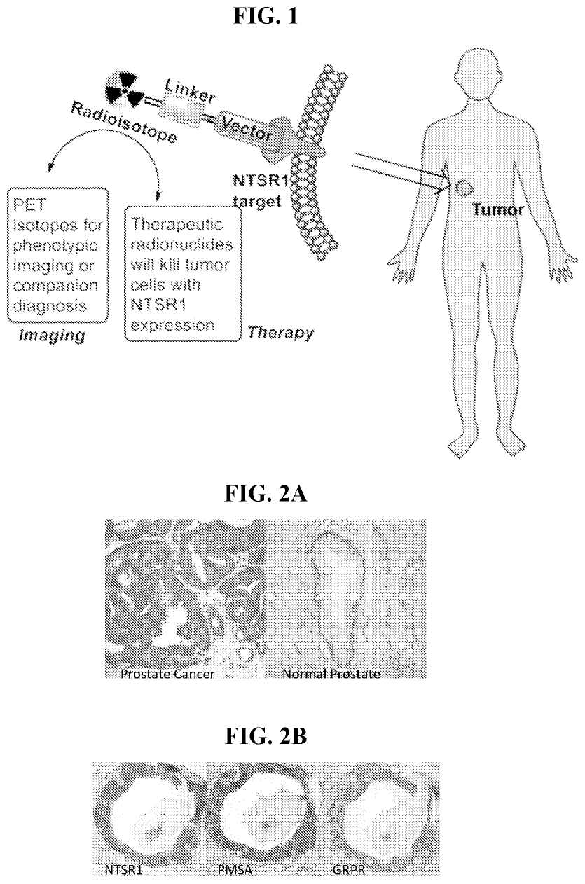

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, 5%, ±1%, ±0.5%, or even ±0.1% of X. A range provided herein for a measurable value may include any other range and/or individual value therein.

Also as used herein, “and/or” refers to and encompasses any and all possible combinations of one or more of the associated listed items, as well as the lack of combinations when interpreted in the alternative (“or”).

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The terms “treat,” or “treating” or “treatment” refer to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, etc., as would be well known in the art.

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

A “treatment effective” amount, “effective amount,” or “therapeutic amount” as used herein is an amount that is sufficient to provide some improvement or benefit to the subject. Alternatively stated, a “treatment effective amount,” “effective amount,” or “therapeutic amount” is an amount that will provide some alleviation, mitigation, decrease or stabilization in at least one clinical symptom in the subject. Those skilled in the art will appreciate that the therapeutic effects need not be complete or curative, as long as some benefit is provided to the subject. The effective amount may vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutic amount in any individual case can be determined by one of ordinary skill in the art by reference to the pertinent texts and literature and/or by using routine experimentation. (See, for example, Remington, The Science and Practice of Pharmacy (20th ed. 2000)).

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, sterile pyrogen-free water and sterile pyrogen-free physiological saline solution.

The term “administering” or “administration” of a composition of the present invention to a subject includes any route of introducing or delivering to a subject a compound to perform its intended function (e.g., for use in PET imaging, e.g., for the guidance of surgery).

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human.

A “subject in need” of the methods of the invention can be any subject known or suspected to have cancer and/or an illness to which imaging and/or surgery may provide beneficial health effects, or a subject having an increased risk of developing the same.

A “sample”, “biological sample”, and/or “ex vivo sample” of this invention can be any biological material, such as a biological fluid, an extract from a cell, an extracellular matrix isolated from a cell, a cell (in solution or bound to a solid support), a tissue, a tissue homogenate, a biopsy, and the like as are well known in the art.

As used herein, by “isolate” or “purify” (or grammatical equivalents) a fragment, it is meant that the fragment is at least partially separated from at least some of the other components in the starting material.

### NTSR1-Specific Ligand

Based on the important function of NTSR1, a corresponding PET agent may serve as a promising prognostic marker useful to identify, within early stage disease patients, those with a bad prognosis. Furthermore, the role of NTS in the growth of experimental tumors may represent a basis for the development of specifically targeted drugs (including therapeutic radionuclide-based agent) to be used together with currently available treatments.

As used herein, the term “neurotensin receptor (NTSR1)-specific ligand” and/or “NTSR1-targeted ligand” refers to a core scaffold structure which includes a NTSR1 targeting ligand, a bifunctional linker, and a radionuclide component (see, e.g., FIG. 9).

The term “neurotensin ligand” refers to a molecule that specifically binds NTSR1.

As used herein, the term “SR-CP-05” refers to a core scaffold structure which includes a region that binds to NTSR1. In some embodiments, a NTSR1-specific ligand of the present invention may comprise the NTS agent SR-CP-05, a linker, and a chelator for binding a radioisotope. In some embodiments, the bond link between the linker (e.g., polyamine) and chelator is an amide or other bond. In some embodiments, the region that binds to NTSR1 is a NTS agent. In one embodiment, the region that binds to NTSR1 is the NTS agent SR-CP-05 and refers to the structure having formula X:

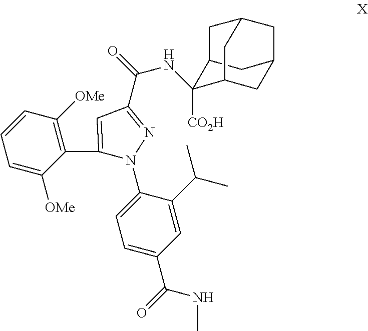 [SMILES: CNC(=O)c1ccc(-n2nc(C(=O)NC3(C(=O)O)C4CC5CC(C4)CC3C5)cc2-c2c(OC)cccc2OC)c(C(C)C)c1]

In some embodiments, the NTSR1-targeted ligand of the present invention may comprise the structure having formula I or a pharmaceutically acceptable salt thereof:

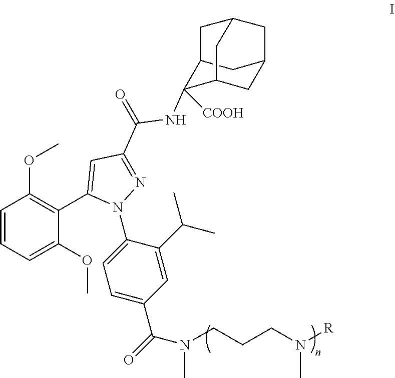

wherein n is 1-3 and R is a chelator.

Non-limiting examples of chelators are NOTA, DOTA, Cross Bridge-Cyclam, Cross Bridge-TE2A, Sarcage and derivatives thereof (the bond link between the linker (e.g., polyamine) and chelator can be amide or other bonds).

In one embodiment, the NTSR1-targeted ligand of the present invention is NT-2PA-CB and comprises the structure having formula Ia or a pharmaceutically acceptable salt thereof:

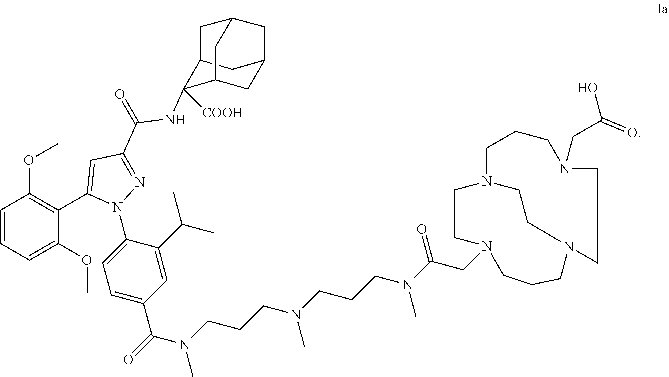 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCCN3CCN(CCCN(CC(=O)O)CC3)CC2)cc1C(C)C]

In one embodiment, the NTSR1-targeted ligand of the present invention is NT-1PA-CB and comprises the structure having formula Ib or a pharmaceutically acceptable salt thereof:

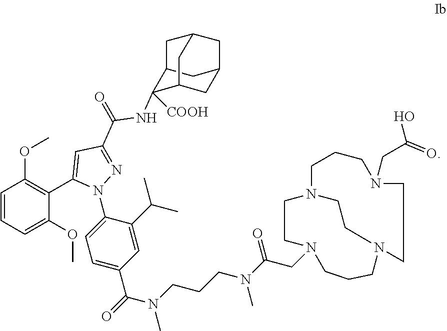 [SMILES: COc1cccc(OC)c1-c1cc(C(=O)NC2(C(=O)O)C3CC4CC(C3)CC2C4)nn1-c1ccc(C(=O)N(C)CCCN(C)C(=O)CN2CCCN3CCN(CCCN(CC(=O)O)CC3)CC2)cc1C(C)C]

In one embodiment, the NTSR1-targeted ligand of the present invention is NT-3PA-DOTA and comprises the structure having formula Ic or a pharmaceutically acceptable salt thereof:

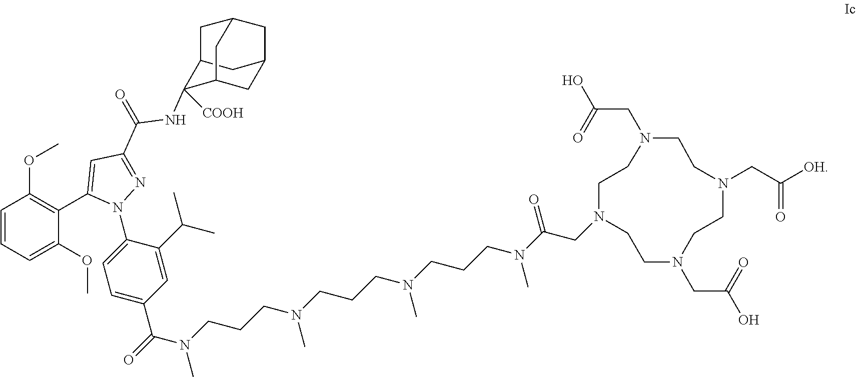 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)CCCN(C)C(=O)CN2CCN(CC(=O)O)CCN(CC(=O)O)CCN(CC(=O)O)CC2)cc1C(C)C]

In one embodiment, the NTSR1-targeted ligand of the present invention is NT-3PA-CB and comprises the structure having formula Id or a pharmaceutically acceptable salt thereof:

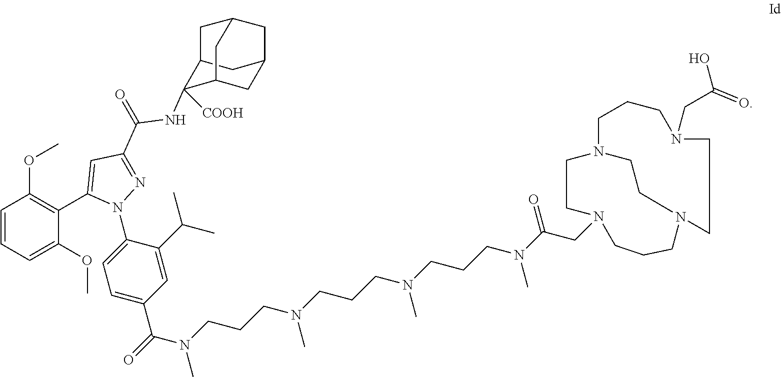 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)CCCN(C)C(=O)CN2CCCN3CCN(CCCN(CC(=O)O)CC3)CC2)cc1C(C)C]

In some embodiments, the NTSR1-targeted ligand of the present invention may comprise the structure having formula II or a pharmaceutically acceptable salt thereof:

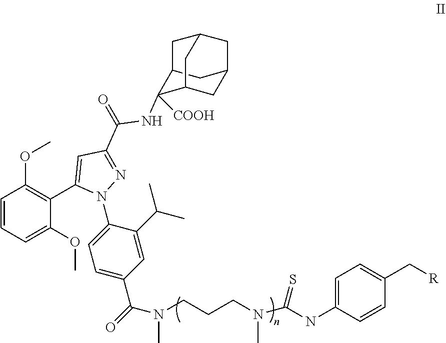

wherein n is 1-3 and R is a chelator.

In one embodiment, the NTSR1-targeted ligand of the present invention is NT-1PA-NOTA and comprises the structure having formula IIa or a pharmaceutically acceptable salt thereof:

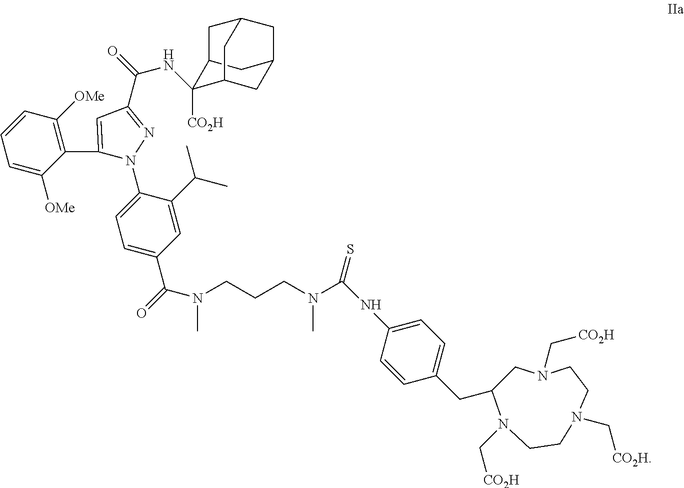 [SMILES: COc1cccc(OC)c1-c1cc(C(=O)NC2(C(=O)O)C3CC4CC(C3)CC2C4)nn1-c1ccc(C(=O)N(C)CCCN(C)C(=S)Nc2ccc(CC3CN(CC(=O)O)CCN(CC(=O)O)CCN3CC(=O)O)cc2)cc1C(C)C]

In one embodiment, the NTSR1-targeted ligand of the present invention is NT-2PA-NOTA and comprises the structure having formula IIb or a pharmaceutically acceptable salt thereof:

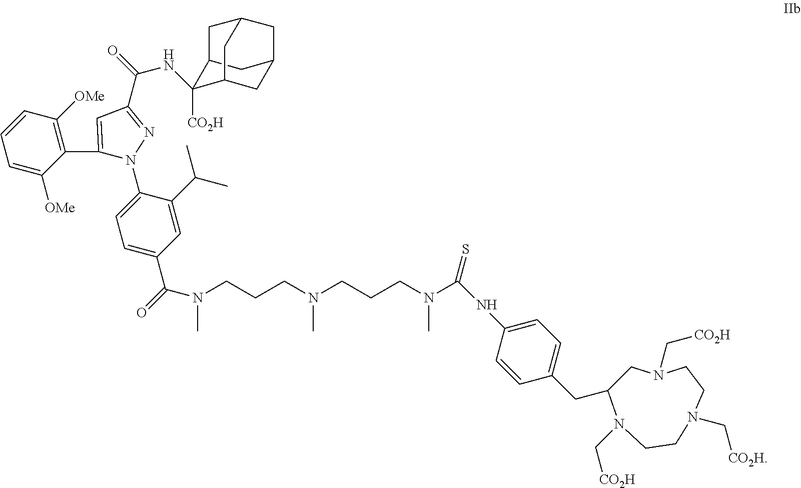 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=S)Nc2ccc(CC3CN(CC(=O)O)CCN(CC(=O)O)CCN3CC(=O)O)cc2)cc1C(C)C]

In some embodiments, the NTSR1-targeted ligand of the present invention may comprise the structure having formula III or a pharmaceutically acceptable salt thereof:

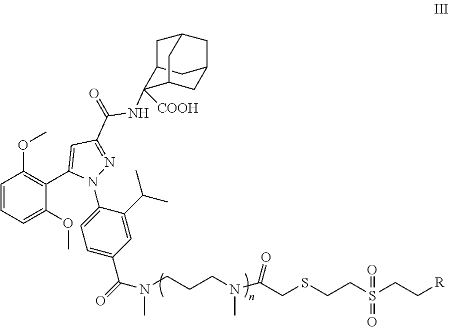

wherein n is 1-3 and R is a chelator.

In one embodiment, the NTSR1-targeted ligand of the present invention is NT-2PA-VS-DOTA and comprises the structure having formula IIIa or a pharmaceutically acceptable salt thereof:

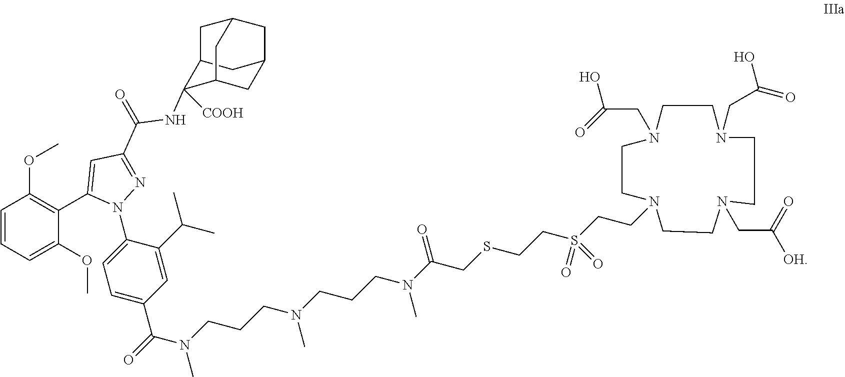 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CSCCS(=O)(=O)CCN2CCN(CC(=O)O)CCN(CC(=O)O)CCN(CC(=O)O)CC2)cc1C(C)C]

In some embodiments, the NT SRI-targeted ligand of the present invention may comprise the structure having formula IV or a pharmaceutically acceptable salt thereof:

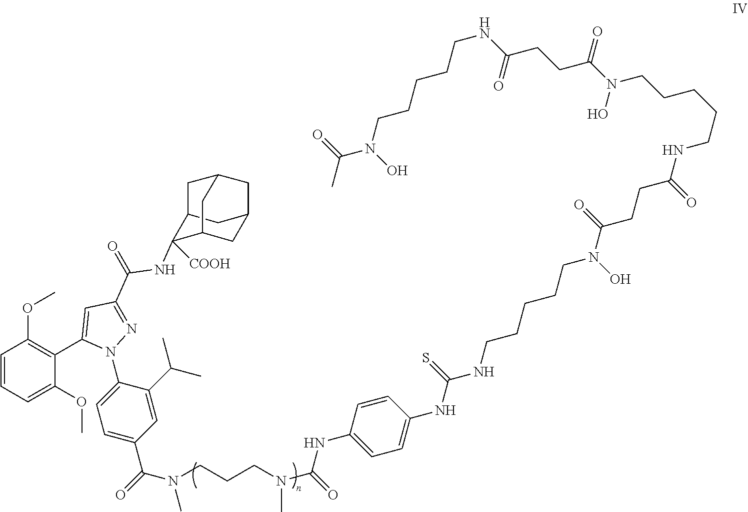

wherein n is 1-3.

In one embodiment, the NTSR1-targeted ligand of the present invention is NT-2PA-Df and comprises the structure having formula IVa or a pharmaceutically acceptable salt thereof:

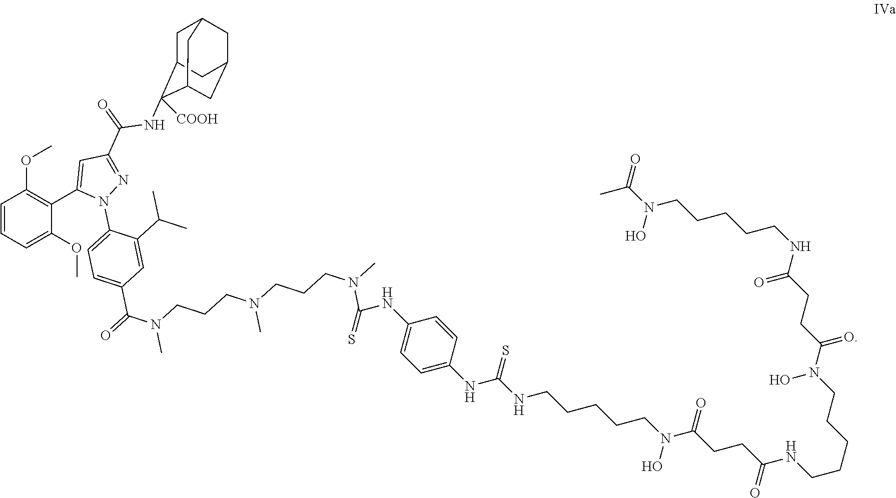 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=S)Nc2ccc(NC(=S)NCCCCCN(O)C(=O)CCC(=O)NCCCCCN(O)C(=O)CCC(=O)NCCCCCN(O)C(C)=O)cc2)cc1C(C)C]

In one embodiment, the NTSR1-targeted ligand of the present invention is NT-1PA-Df and comprises the structure having formula IVb or a pharmaceutically acceptable salt thereof:

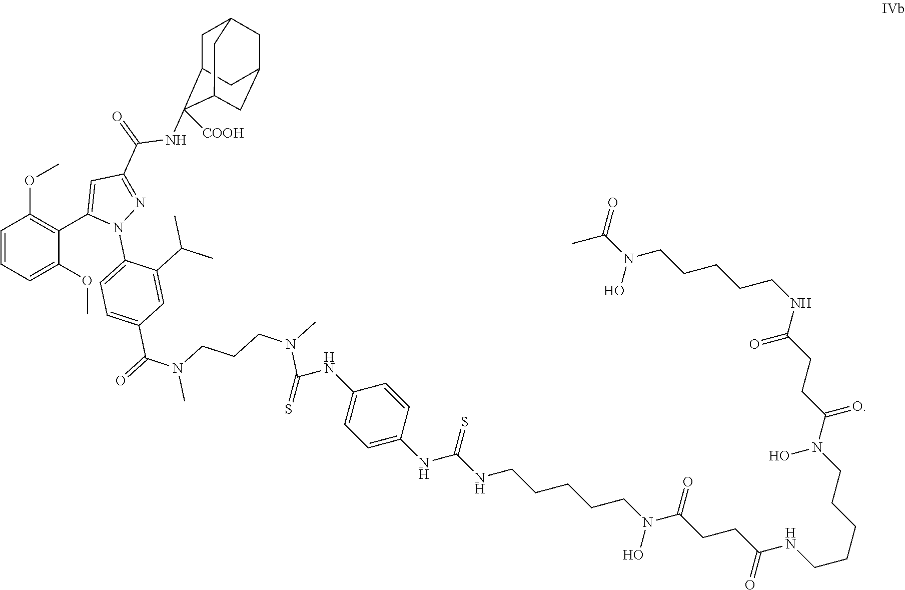 [SMILES: COc1cccc(OC)c1-c1cc(C(=O)NC2(C(=O)O)C3CC4CC(C3)CC2C4)nn1-c1ccc(C(=O)N(C)CCCN(C)C(=S)Nc2ccc(NC(=S)NCCCCCN(O)C(=O)CCC(=O)NCCCCCN(O)C(=O)CCC(=O)NCCCCCN(O)C(C)=O)cc2)cc1C(C)C]

In some embodiments, the NT SRI-targeted ligand of the present invention may comprise the structure having formula V or a pharmaceutically acceptable salt thereof:

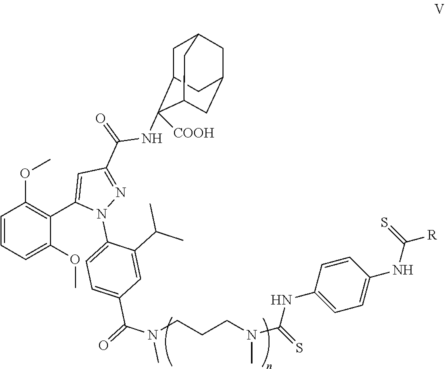

wherein n is 1-3 and R is a chelator.

In one embodiment, the NTSR1-targeted ligand of the present invention is NT-1PA-sarcage and comprises the structure having formula Va or a pharmaceutically acceptable salt thereof:

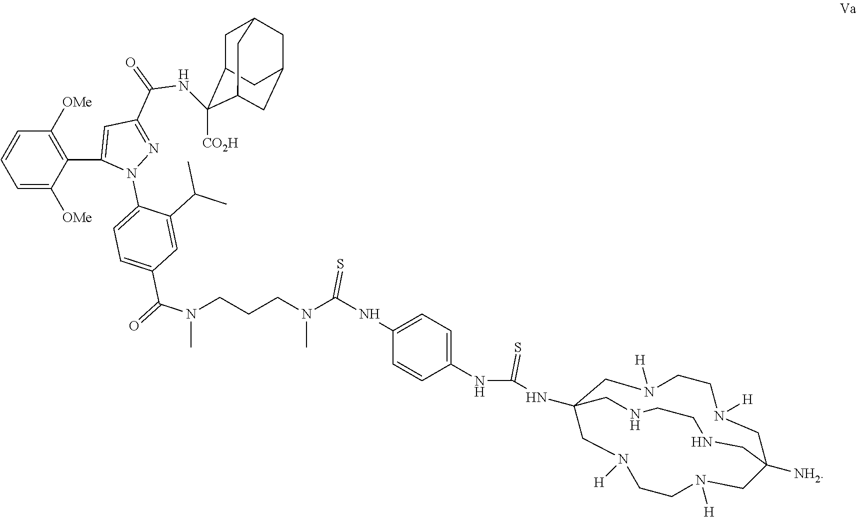 [SMILES: COc1cccc(OC)c1-c1cc(C(=O)NC2(C(=O)O)C3CC4CC(C3)CC2C4)nn1-c1ccc(C(=O)N(C)CCCN(C)C(=S)Nc2ccc(NC(=S)NC34CNCCNCC(N)(CNCCNC3)CNCCNC4)cc2)cc1C(C)C]

In one embodiment, the NTSR1-targeted ligand of the present invention is NT-1PA-sarcage and comprises the structure having formula Vb or a pharmaceutically acceptable salt thereof:

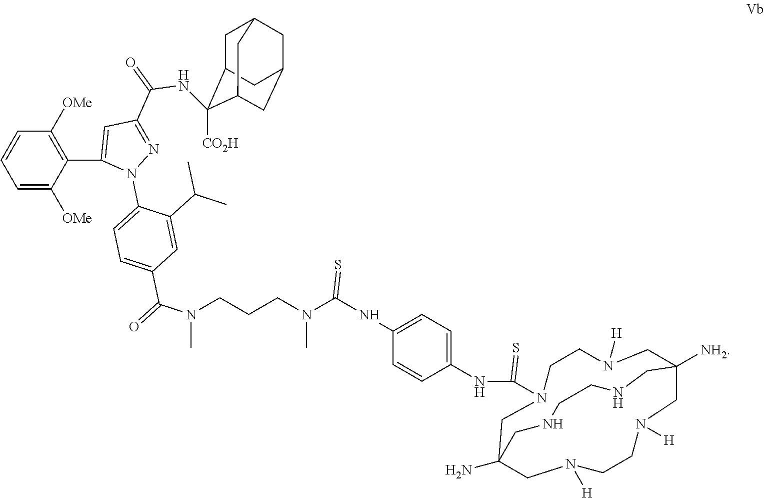 [SMILES: COc1cccc(OC)c1-c1cc(C(=O)NC2(C(=O)O)C3CC4CC(C3)CC2C4)nn1-c1ccc(C(=O)N(C)CCCN(C)C(=S)Nc2ccc(NC(=S)N3CCNCC4(N)CNCCNCC(N)(CNCCNC4)C3)cc2)cc1C(C)C]

In one embodiment, the NTSR1-targeted ligand of the present invention is NT-2PA-sarcage and comprises the structure having formula Vc or a pharmaceutically acceptable salt thereof:

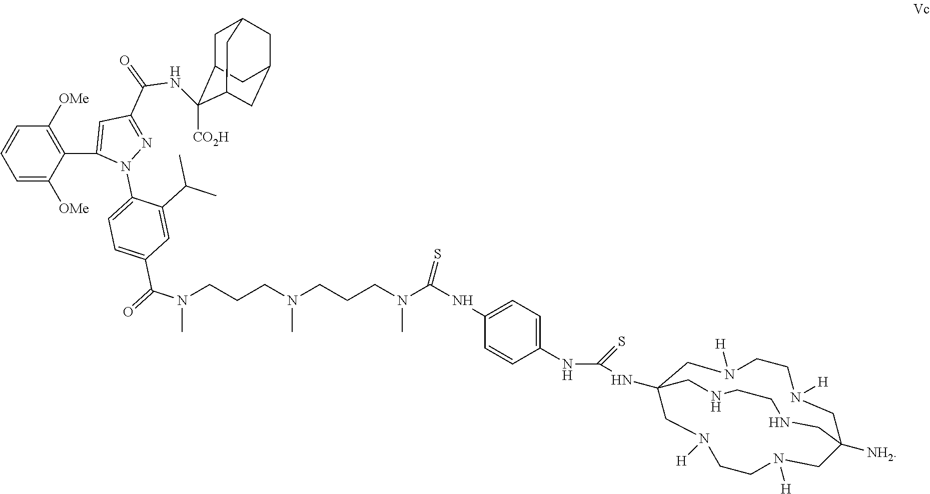 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=S)Nc2ccc(NC(=S)NC34CNCCNCC(N)(CNCCNC3)CNCCNC4)cc2)cc1C(C)C]

In one embodiment, the NTSR1-targeted ligand of the present invention is NT-2PA-sarcage and comprises the structure having formula Vd or a pharmaceutically acceptable salt thereof:

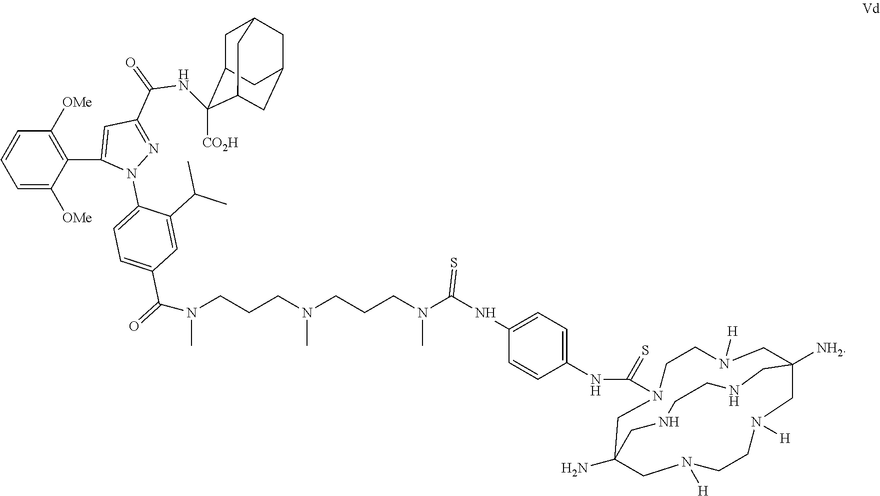 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=S)Nc2ccc(NC(=S)N3CCNCC4(N)CNCCNCC(N)(CNCCNC4)C3)cc2)cc1C(C)C]

In some embodiments, the NT SRI-targeted ligand of the present invention may comprise the structure having formula VI or a pharmaceutically acceptable salt thereof:

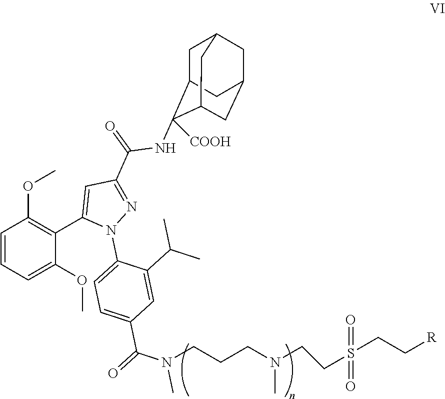

wherein n is 1-3 and R is a chelator.

In one embodiment, the NTSR1-targeted ligand of the present invention is NT-2PA-VS-Sarcage and comprises the structure having formula VIa or a pharmaceutically acceptable salt thereof:

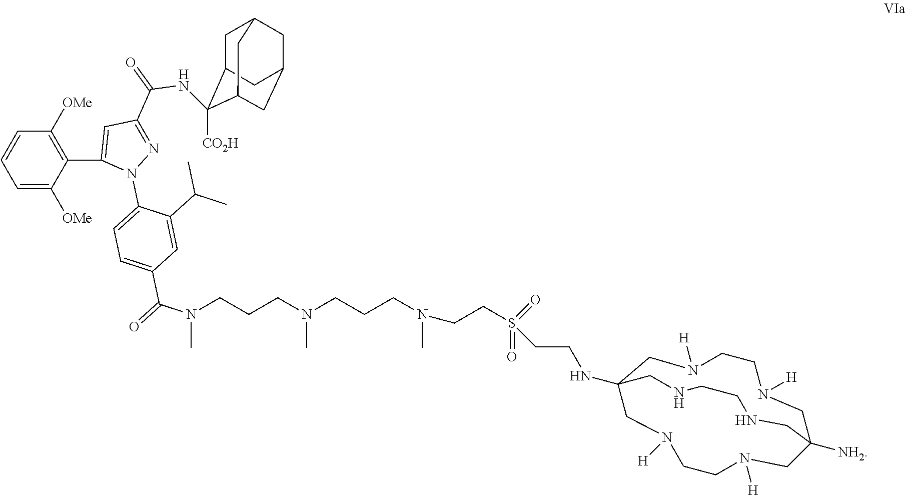 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)CCS(=O)(=O)CCNC23CNCCNCC(N)(CNCCNC2)CNCCNC3)cc1C(C)C]

In some embodiments, the NTSR1-targeted ligand of the present invention may comprise the structure having formula VII or a pharmaceutically acceptable salt thereof:

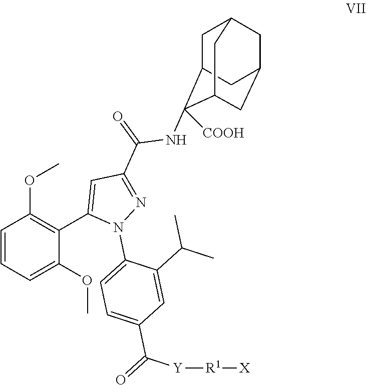

wherein Y is a bond or is selected from PEG, di-vinyl sulfone, di-halogenated alkyls, phenylene isothiocyanate, p-phenylene diisothiocyanate, 1,4-diazinane, and phosphates; X is H or LR²; L is selected from PEG, di-vinyl sulfone, di-halogenated alkyls, phenylene isothiocyanate, p-phenylene diisothiocyanate, 1,4-diazinane, and phosphates; and R¹ and R² are chelators.

In one embodiment, the NTSR1-targeted ligand of the present invention is NT-sarcage and comprises the structure having formula VIIa or a pharmaceutically acceptable salt thereof:

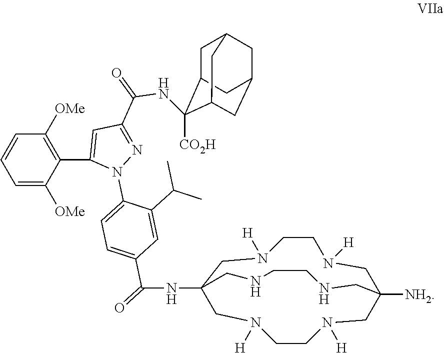 [SMILES: COc1cccc(OC)c1-c1cc(C(=O)NC2(C(=O)O)C3CC4CC(C3)CC2C4)nn1-c1ccc(C(=O)NC23CNCCNCC(N)(CNCCNC2)CNCCNC3)cc1C(C)C]

In one embodiment, the NTSR1-targeted ligand of the present invention is NT-sarcage and comprises the structure having formula VIIb or a pharmaceutically acceptable salt thereof:

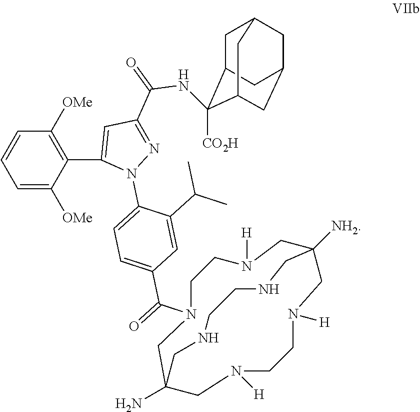 [SMILES: COc1cccc(OC)c1-c1cc(C(=O)NC2(C(=O)O)C3CC4CC(C3)CC2C4)nn1-c1ccc(C(=O)N2CCNCC3(N)CNCCNCC(N)(CNCCNC3)C2)cc1C(C)C]

In one embodiment, the NTSR1-targeted ligand of the present invention is NT-CB and comprises the structure having formula VIIc or a pharmaceutically acceptable salt thereof:

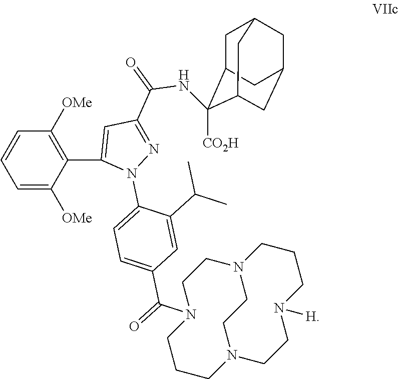

In one embodiment, the NTSR1-targeted ligand of the present invention is NT-CB-CA and comprises the structure having formula VIId or a pharmaceutically acceptable salt thereof:

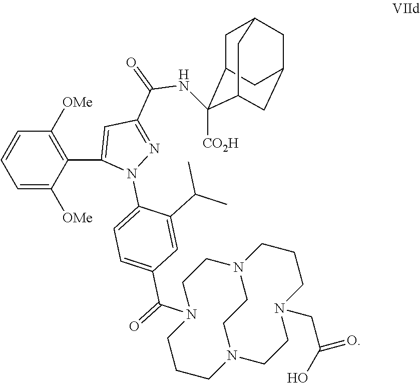 [SMILES: COc1cccc(OC)c1-c1cc(C(=O)NC2(C(=O)O)C3CC4CC(C3)CC2C4)nn1-c1ccc(C(=O)N2CCCN3CCN(CCCN(CC(=O)O)CC3)CC2)cc1C(C)C]

In one embodiment, the NTSR1-targeted ligand of the present invention is NT-CB-CB and comprises the structure having formula VIIe or a pharmaceutically acceptable salt thereof:

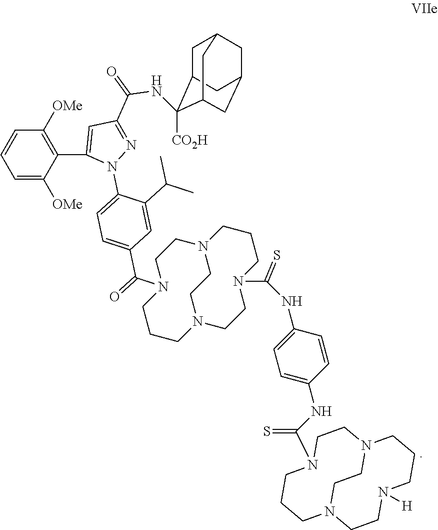 [SMILES: COc1cccc(OC)c1-c1cc(C(=O)NC2(C(=O)O)C3CC4CC(C3)CC2C4)nn1-c1ccc(C(=O)N2CCCN3CCN(CCCN(C(=S)Nc4ccc(NC(=S)N5CCCN6CCNCCCN(CC6)CC5)cc4)CC3)CC2)cc1C(C)C]

In one embodiment, the NTSR1-targeted ligand of the present invention is NT-CB-NOTA and comprises the structure having formula VIIf or a pharmaceutically acceptable salt thereof:

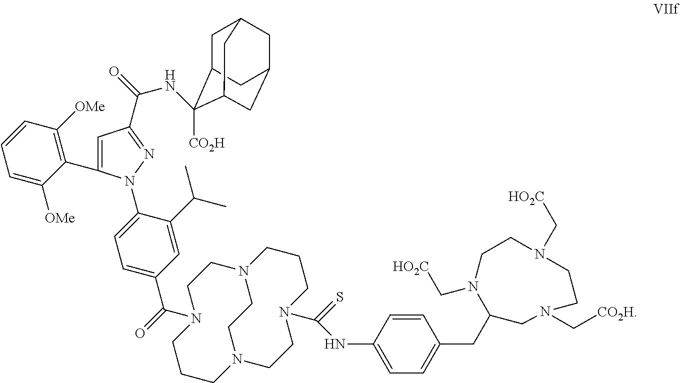 [SMILES: COc1cccc(OC)c1-c1cc(C(=O)NC2(C(=O)O)C3CC4CC(C3)CC2C4)nn1-c1ccc(C(=O)N2CCCN3CCN(CCCN(C(=S)Nc4ccc(CC5CN(CC(=O)O)CCN(CC(=O)O)CCN5CC(=O)O)cc4)CC3)CC2)cc1C(C)C]

In one embodiment, the NTSR1-targeted ligand of the present invention is NT-CB-DOTA and comprises the structure having formula VIIg or a pharmaceutically acceptable salt thereof:

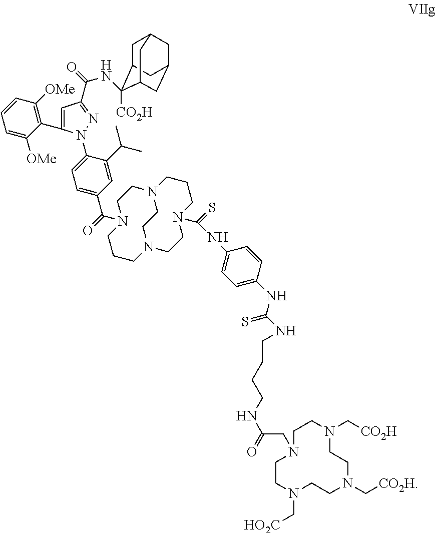 [SMILES: COc1cccc(OC)c1-c1cc(C(=O)NC2(C(=O)O)C3CC4CC(C3)CC2C4)nn1-c1ccc(C(=O)N2CCCN3CCN(CCCN(C(=S)Nc4ccc(NC(=S)NCCCCNC(=O)CN5CCN(CC(=O)O)CCN(CC(=O)O)CCN(CC(=O)O)CC5)cc4)CC3)CC2)cc1C(C)C]

In one embodiment, the NTSR1-targeted ligand of the present invention is NT-CB-sarcage and comprises the structure having formula VIIh or a pharmaceutically acceptable salt thereof:

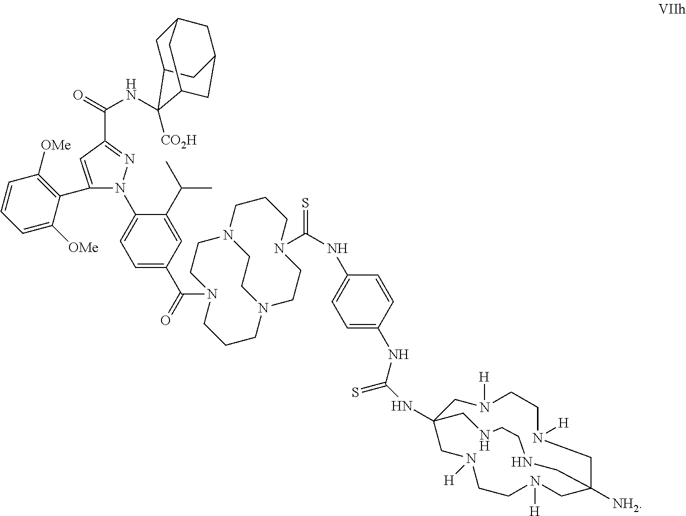 [SMILES: COc1cccc(OC)c1-c1cc(C(=O)NC2(C(=O)O)C3CC4CC(C3)CC2C4)nn1-c1ccc(C(=O)N2CCCN3CCN(CCCN(C(=S)Nc4ccc(NC(=S)NC56CNCCNCC(N)(CNCCNC5)CNCCNC6)cc4)CC3)CC2)cc1C(C)C]

In one embodiment, the NTSR1-targeted ligand of the present invention is NT-CB-sarcage and comprises the structure having formula VIIi or a pharmaceutically acceptable salt thereof:

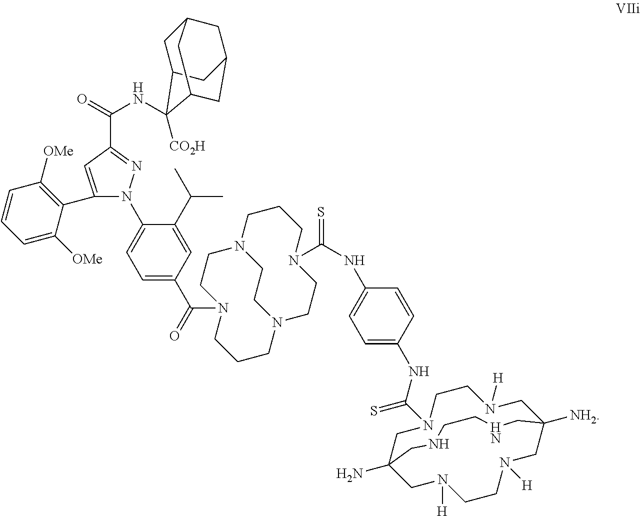 [SMILES: COc1cccc(OC)c1-c1cc(C(=O)NC2(C(=O)O)C3CC4CC(C3)CC2C4)nn1-c1ccc(C(=O)N2CCCN3CCN(CCCN(C(=S)Nc4ccc(NC(=S)N5CCNCC6(N)CNCCNCC(N)(CNCCNC6)C5)cc4)CC3)CC2)cc1C(C)C]

In some embodiments, the NTSR1-specific ligand may comprise a radioisotope. The radioisotope may be one suitable for imaging and/or suitable for therapeutic use. In other embodiments, the radioisotope is selected from ⁶⁴Cu, ⁶⁷Cu, ⁶⁸Ga, ¹⁷⁷Lu, ¹⁸F, ⁹⁰Y, ²²⁵Ac, ²²⁷Th, ²²³Ra, ²¹³Bi, ²¹¹At, ²¹²Pb, ²¹²Bi, ²³⁰U, ²²⁶Th, and ¹⁴⁹Tb. In some embodiments, the NTSR1-specific ligand has a radio purity of at least 95% (e.g., 95%, 96%, 97%, 98%, 99%, or higher).

In some embodiments, the NTSR1-specific ligand may comprise one or more chelators selected from NOTA, DOTA, Cross Bridge-Cyclam, Cross Bridge-TE2A, DF chelators, DiAm-Sarcage and derivatives thereof. In other embodiments, the NTSR1-specific ligand may comprise one or more linkers selected from NOTA, DOTA, Cross Bridge-Cyclam, Cross Bridge-TE2A, Sarcage, and derivatives thereof, polyamines (e.g., urea), PEG, di-vinyl sulfones, di-halogenated alkyls, phenylene isothiocyanate, p-phenylene diisothiocyanate, 1,4-diazinane, phosphates, and combinations thereof. In some embodiments, the NTSR1-specific ligand may comprise the NTS ligand, SR-CP-05.

One aspect of the invention relates to a positron emission tomography (PET) probe comprising the NTSR1-specific ligand. In one embodiment, the PET probe has a radioisotope, optionally wherein the radioisotope is ⁶⁴Cu or ⁶⁸Ga.

Another aspect of the invention relates to a therapeutic agent comprising the NTSR1-specific ligand. In one embodiment, the therapeutic agent comprises a radioisotope, optionally wherein the radioisotope is ⁶⁷Cu, ¹⁷⁷Lu, ¹⁸F, ⁹⁰Y, ²²⁵Ac, ²²⁷Th, ²²³Ra, ²¹³Bi, ²¹¹At, ²¹²Pb, ²¹²Bi, ²³⁰U, ²²⁶Th, and ¹⁴⁹Tb.

### ¹⁸F Labeled NTSR1-Targeted Ligand

In some embodiments, the NTSR1-targeted ligand may be ¹⁸F labeled. ¹⁸F may be the preferred PET isotope from an imaging point of view. Despite recent progress on ⁶⁸Ga labeled PET agents, recent shortages of the ⁶⁸Ga isotope and the greatly increased cost of ⁶⁸Ga/⁶⁸Ge generators justified the need for investigating alternative agents. Compared with ⁶⁸Ga labeled agents, ¹⁸F labeled analogs may have various advantages including availability of Curies of ¹⁸F from existing medical cyclotrons; the ability to be produced in multi-doses from a single run; increased half-life that will facilitate commercialization, the capability to image late time points; and the potential improved contrast/resolution due to lower positron energy and higher positron percentage. ¹⁸F labeled PET agents targeting NTSR1 may comprise a chelator, which would allow integration of PET imaging with radiometal based therapy.

Traditional labeling methods may be employed for ¹⁸F radiolabeling, which includes amide bond formation, oxime formation (reaction of 4-¹⁸F-fluorobenzaldehyde (¹⁸F-FBA) with hydroxylamine derived NT peptides, Michael addition (¹⁸F-labeled maleimide synthon with thiolated NT peptides), or click chemistry (¹⁸F-labeled alkyne with azide modified NT peptides). However, most of these traditional methods generally require several time-consuming radiosynthetic steps resulting in overall low labeling yield.

A vinyl sulfone based labeling method can be used to create a unique thiol-reactive ¹⁸F-labeling method based on vinyl sulfone for facile preparation of ¹⁸F-labeled NT agent. This method can use ¹⁸F-DEG-VS (shown below) for ¹⁸F labeling of thiolated NTSR1 binding ligands as well.

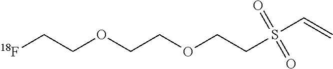

A tetrazine trans-cyclooctene based labeling method can also be used to label the potent NTSR1 binding ligands with ¹⁸F. In order to obtain reasonable labeling yield of the final product, excess amounts of ligands (in the range of 200 μg-2 mg) are generally required using the above labeling methods. However, some embodiments may have low synthetic yield at the beginning. The amount of ligands obtained may be too small for ¹⁸F labeling (for example, only 50-100 μg of the ligands were obtained). In some embodiments, the ¹⁸F labeled probe is tested for its imaging property in vivo first, prior to optimization of synthetic conditions. A fast and ultra-efficient method for generating 18F labeled-probes may be used, which greatly expedites the tracer development process. An ultra efficient ¹⁸F labeling method based on tetrazine ligation (namely TTCO ligation) may be used. The tetrazine ligation is a bioorthogonal reaction. The reaction is based on the inverse-electron-demand Diels-Alder reactions between trans-cyclooctene and diaryltetrazines, which produces N2 as the only byproduct upon subsequent retro-[4+2]cycloaddition.

¹⁸F-labeled trans-cyclooctenes can be obtained in high radiochemical yield (71%) in hundreds of mCis. The reaction between TCOs and tetrazine modified ligands may proceed with exceptionally fast rates, making it an effective method for creating ¹⁸F-labeled probes within seconds at low micromolar concentrations. Using peptide based ligands, various NTSR1 targeted PET agents can be used based on TTCO ligation. NTSR1 binding ligands can also be functionalized with tetrazine through other reactions (for example, thiolated ligands may react with tetrazine-Mal to obtain tetrazine-NT). More than ten ¹⁸F labeled probes may be easily obtained in high yield within one day using this ultra-efficient labeling method.

The ability to combine imaging and therapy agents into one molecule may minimize the discrepancy between imaging agent and therapy agent. ¹⁸F-labeled NTSR1-targeting agents also bearing radio metal chelators may be used for radionuclide based therapy applications.

In brief, new NTSR1-targeting agents may be constructed with ¹⁸F labeling based on the TTCO ligation method and SR-CP-05 based ligands. A metal chelator may also be included in the design for therapeutic nuclide chelation. This design can take advantage of favorable properties of ¹⁸F from a commercial point of view, and make the imaging and therapy agent have the same backbone structure. Tetrazine of the SR-CP-05 derivatives can be reacted with ¹⁸F-TCOs to obtain the PET agent. Other clickable methods can also be used. ¹⁸F-labeled agents with and without metal (chelated to DOTA) can be prepared and may have an isolation yield of >5%.

The NTSR1 binding affinity can be evaluated using H1299 cells. Selected agents may have an IC₅₀<20 nM. The non-specific binding may be less than 30%. The serum and metabolic stability of the PET probes can be evaluated by incubation in rat serum. Selected agents can have a serum stability of >80% at 1 hr post incubation. The tumor targeting efficacy and in vivo kinetics of ¹⁸F-labeled NTSR1 probes can be tested in a subcutaneous H1299 xenograft model. The time points may be 0.5, 1, 2, and 4 hr post injection. For each small animal PET scan, 3D regions of interests (ROIs) can be drawn over tumor, heart, liver, kidneys and muscle on decay-corrected whole-body coronal images to obtain an imaging ROI derived percentage ID per gram of tissue (% ID/g). At the end of each scan, the animal may be sacrificed and the accuracy of non-invasive PET quantification can be assessed by direct tissue sampling. The target specificity of the tracer in vivo can be confirmed using a blocking experiment. The selected agent may have tumor uptake >5% ID/g. Non-specific binding may be less than 40% in vivo.

The PET probe can be selected based on the following criteria: labeling yield >5% (>15 mCi product can be obtained for clinical translation), good tumor uptake (>5% ID/g at 1 hr post injection), optimal tumor/background contrast (tumor/liver >1, tumor/kidney >1, tumor/muscle >10 at 1 hr post injection), good target specificity as confirmed by a blocking experiment (uptake can be decreased by >60% in block group). SR-CP-05 based PET probes can be compared side by side with ⁶⁸Ga or ⁶⁴Cu based agents for NTSR1 imaging.

Bifunctional linkers can be used to modify the radiotracer's pharmacokinetics and decrease uptake in the liver. For example, PEGylation can be performed to increase the water solubility of the imaging probe to reduce its liver uptake. Both short and long PEG (PEGₙ, n is 2-10) can be used for imaging probe construction. Glycan based linkers can also be used to increase water solubility. After bifunctional linkers are introduced to the peptides, their NTSR-1 binding affinity can be determined.

The PET probes and/or therapeutic agents can be compared side by side with ⁶⁸Ga or ⁶⁴Cu based agents in order to fully characterize their ability to quantify NTSR1 expression in vivo. The imaging can be carried out using various lung cancer models including H1299, H1975, H23, H226, and H460. All of these are NSCLC cells that are NTSR1 positive. Notably, H1299, H1975, and H23 originated from adenocarcinoma NSCLC, whereas H226 and H460 originated from squamous cell carcinoma and large cell carcinoma, respectively. For comparison, H358 and H1973 (NTSR1 negative NSCLC) can be used as negative controls. Western blot and FACS analyses of the above lung cancer tumors can be performed to determine the NTSR1 expression level. Corresponding xenograft models can be imaged with the PET probes and/or therapeutic agents. ROI can be drawn to obtain the tumor uptake as percentage ID per gram of tissue (% ID/g). Western analysis of tumor tissues can be performed to characterize the relative expression level of NTSR1, which can then be correlated with the PET derived tumor uptake using GraphPad software. To qualitatively evaluate the NTSR1 expression in each tumor tissue, immunofluorescence staining can be performed on H1299, H1975, H23, H226, and H460 xenograft models. DAPI and CD31 staining can also be performed. Through these studies, the best imaging agent for NTSR1 can be chosen for further evaluation in non-human primates. In some embodiments, the selection criteria may be: tumor/liver >1, tumor/kidney >1, tumor/muscle >10 at 1 hr post injection in NTSR1 positive tumor models. Tumor uptake may correlate with NTSR1 expression in different tumor types. The tumor uptake value can be considered together with contrast. If the contrast shows no significant difference, the one with higher tumor uptake can be selected.

The PET probes and/or therapeutic agents can be evaluated in non-human primates (e.g., Rhesus Macaque). After injection with radiolabeled agent i.v., a 2-hour dynamic scan can be performed. A static scan can also be performed at 3 hr if the agent is labeled with ⁶⁸Ga; 4 hr if the agent is labeled with ¹⁸F; and 4 and 24 hr if the agent is labeled with ⁶⁴Cu. The PET probes and/or therapeutic agents may allow (1) characterization of the pharmacokinetics, biodistribution, and metabolic stability of radiolabeled PET agent following brief i.v. infusion; (2) estimation of the dosimetry data; (3) designation of a clinical PET/CT scan protocol, including injection dose and scan time points. In detail, Rhesus Macaque (NHP) can be maintained on 1.4-4% isoflurane inhalation anesthesia and artificial ventilation. Two venous catheters can be applied, one for tracer administration and one for sampling of blood radioactivity concentration. A CT transmission scan can be obtained. Then, radiolabeled PET agent targeting NTSR1 (3-5 mCi) can be given i.v. and a 120-min dynamic PET scan can be performed. The scans can be performed with and without fasting to compare the uptake and contrast difference. Serial venous blood samples (0.2-0.5 ml) can be drawn before and at 0.5, 5, 30, 60, and 90 min post injection to determine metabolic stability and blood uptake. Body temperature, heart rate, ECG, pCO₂, pO₂, SaO₂ and blood pressure can be monitored throughout the study. A urine specimen for IPLC metabolite analysis can be collected at the end of the whole body scan. PET scan data can be analyzed, including volumetric region of interest (ROI) analysis and extraction of tissue time-activity-curves (TACs) and steady-state standardized uptake values (SUVs); quantitative analysis of plasma TACs and HPLC data to determine TACs for circulating PET agent and its metabolites vs. time; and calculated cumulated activities for normal organs/tissues.

Blood plasma and urine samples can be assayed for radiolabeled agent and labeled metabolites. The blood samples can be collected and immediately centrifuged for 5 min at 14,000 rpm. Then, 50% TFA in 100 μL of PBS can be added to the upper serum solution, followed by centrifugation for 5 min. The upper solution can be injected for HPLC analysis. Urine can be filtered, and then used for HPLC analysis.

The distribution of the absorbed radiation dose can be calculated according to the MIRD method, which assumes that the integrated activity is known for each of the source organs. Observed source organs where the PET agent may be concentrated include the urinary bladder, kidneys, and liver. Other organs for which anatomic boundaries can be identified using a combination of the PET scan, the attenuation scan, and the comparison CT scan can be used as additional source organs for completion (brain, lower large intestine, stomach, blood, heart wall, lung, pancreas, red marrow, spleen). Organs within which no PET uptake above background is observed and for which boundaries cannot be delineated can be treated as background and assigned the remainder level of cumulated activity.

Non-human primates can be closely monitored for the development of toxicity. Metabolic studies can be performed, including blood chemistry profile (electrolytes, glucose, calcium, phosphorous, magnesium, bilirubin, albumin, total proteins, AST, ALT, ALP) to assess potential liver and kidney function changes.

Statistically significant data can be obtained by using 10 animals per group for the comparison of the agents. Statistical differences between imaging groups at different time points and from different methods can be assessed by ANOVA and Student's t-test for unpaired data between two groups. The level of significance can be set at P<0.05. If P >0.05, Tukey-Kramer multiple comparisons post-tests can be performed. Power calculations are based on the assumption that a meaningful decrease in imaging signal (or tumor size), which can be designed to detect using a one-tailed (two-group) t-test with alpha set at 0.05, is 20%. The intra-group standard deviation can be set at 15% of the mean value. With a group size of 10 animals per group, the power may be 0.89. With a smaller inter-group decrease in imaging signal (or tumor size) of 10%, there may be adequate power (0.70) with 10 animals per group.

### Methods of Use

Additional aspects of the invention relate to methods of using the compounds of the invention for imaging, diagnosing, and/or guidance of treatment of a NTSR1-positive cancer or a disorder of a NTSR1-positive tissue. In some embodiments, the NTSR1-positive cancer is a cancer of a tissue selected from prostate, liver, kidney, spleen, bladder, parotid gland, lacrimal gland, submandibular glands, small intestine, nasal mucosa, esophageal mucosa, vocal cords, gallbladder, biliary tract, trachea, lung, breast, mediastinal lymph nodes, axillary lymph nodes, inguinal lymph nodes, gynecomastia, sympathetic ganglia, and any combination thereof.

One aspect of the invention relates to a method of carrying out a PET scan on a subject, comprising administering to the subject a ligand, probe, and/or composition of the present invention.

Another aspect of the invention provides a method of imaging tissue comprising a NTSR1-positive cancer in a subject, comprising administering to the subject a ligand, probe, and/or composition of the present invention.

Another aspect of the invention provides a method of imaging prostate cancer in a subject, comprising administering to the subject a ligand, probe, and/or composition of the present invention.

Another aspect of the invention provides a method of imaging lung cancer in a subject, comprising administering to the subject a ligand, probe, and/or composition of the present invention.

Another aspect of the invention provides a method of identifying NTSR1-positive cancer tissue in a subject, comprising carrying out a PET scan on the subject using a ligand, probe, or composition of the present invention, wherein the PET scan identifies the presence of NTSR1-positive cancer tissue.

Another aspect of the invention provides a method of treating NTSR1-positive cancer tissue in a subject, comprising administering the ligand, therapeutic agent, or composition of the present invention to the subject.

Another aspect of the invention provides a method of removing NTSR1-positive cancer tissue in a subject, comprising: carrying out a PET scan on the subject using a ligand, probe, and/or composition of the present invention, wherein the PET scan identifies the presence of NTSR1-positive cancer tissue; and surgically excising the identified NTSR1-positive cancer tissue, thereby removing the NTSR1-positive cancer tissue.

In some embodiments, the subject has or is at risk for or suspected to have or develop a cancer of a tissue selected from prostate, liver, kidney, spleen, bladder, parotid gland, lacrimal gland, submandibular glands, small intestine, nasal mucosa, esophageal mucosa, vocal cords, gallbladder, biliary tract, trachea, lung, breast, mediastinal lymph nodes, axillary lymph nodes, inguinal lymph nodes, gynecomastia, sympathetic ganglia, and any combination thereof.

Another aspect of the invention provides a method of determining the suitability of a subject with NTSR1-positive cancer or a subject at risk for or suspected to have or develop NTSR1-positive cancer for surgical removal of cancer tissue, comprising (a) carrying out a PET scan on the subject using a ligand, probe, and/or composition of the present invention, wherein the PET scan identifies the presence of NTSR1-positive cancer tissue; and (b) identifying the presence of NTSR1-positive cancer tissue, wherein the presence of NTSR1-positive cancer tissue indicates suitability of the subject for surgical removal of cancer tissue.

Another aspect of the invention provides a method of treating NTSR1-positive cancer in a subject, comprising predicting the suitability of a subject with NTSR1-positive cancer or a subject at risk for or suspected to have or develop NTSR1-positive cancer to surgical removal of cancer tissue by carrying out a PET scan on the subject using a ligand, probe, or composition of the present invention, wherein the PET scan identifies the presence of NTSR1-positive cancer tissue, and treating the NTSR1-positive cancer based on the results of the PET scan.

In some embodiments, treating the NTSR1-positive cancer comprises administering the ligand, therapeutic agent, or composition of the invention. The treatment may be in addition to or instead of standard cancer treatments, including chemotherapy, immunotherapy, radiotherapy, and surgery.

Another aspect of the invention provides a method of treating a disorder of a NTSR1-positive tissue in a subject, comprising determining the suitability of a subject with the disorder or a subject at risk for or suspected to have or develop the disorder to treatment thereof by carrying out a PET scan on the subject using a ligand, probe, or composition of the present invention, wherein the PET scan identifies the presence of NTSR1-positive tissue, and treating the disorder based on the results of the PET scan.

In some embodiments, treating the disorder comprises administering the ligand, therapeutic agent, or composition of the invention. In some embodiments, the disorder is a cancer from a tissue selected from prostate, liver, kidney, spleen, bladder, parotid gland, lacrimal gland, submandibular glands, small intestine, nasal mucosa, esophageal mucosa, vocal cords, gallbladder, biliary tract, trachea, lung, breast, mediastinal lymph nodes, axillary lymph nodes, inguinal lymph nodes, gynecomastia, sympathetic ganglia, any combination thereof.

In some embodiments, the subject is a pre-operative subject. In other embodiments, the subject is an intra-operative subject (e.g., wherein the subject is undergoing surgery).

In some embodiments, treating a disorder of the present invention may comprise surgically excising at least a portion of the identified NTSR1-positive tissue and/or administering an anticancer therapeutic agent such as, e.g., a ligand, therapeutic agent, or composition of the NTSR1-targeted ligands, a chemotherapeutic agent, an immunotherapeutic agent, or any combination thereof.

In some embodiments of the methods of the invention, carrying out a PET scan on the subject using the ligand, probe, and/or composition of the present invention may comprise administering about 1 to about 15 mCi of the ligand, probe, and/or composition.

In some embodiments, the administering is via intravenous injection. In some embodiments, the ligand, PET probe, and/or composition has a tumor to muscle ratio (T/M) of at least 3 or higher at about 1 hour post injection, optionally a tumor to muscle ratio (T/M) of at least 10 or higher at about 1 hour post injection. In some embodiments, the ligand, probe, therapeutic agent, and/or composition clears major organs after 3 hours or less. In some embodiments, the major organs comprise one or more organ(s) selected from liver, kidney, muscle, and any combination thereof.

### Subjects, Pharmaceutical Formulations, and Modes of Administration

The NTSR1-targeted ligands according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, rodents, lagomorphs, etc. Human subjects include neonates, infants, juveniles and adults.

In particular embodiments, the present invention provides a pharmaceutical composition comprising a NTSR1-targeted ligand of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

A further aspect of the invention is a method of administering the NTSR1-targeted ligand to subjects. Administration of the ligand according to the present invention to a human subject or an animal in need thereof can be by any means known in the art. Optionally, the NTSR1-targeted ligand is delivered in a treatment effective dose in a pharmaceutically acceptable carrier.

Dosages of the NTSR1-targeted ligand to be administered to a subject depend upon the mode of administration, the disease or condition to be detected, treated, and/or prevented, the individual subject's condition, the particular NTSR1-targeted ligand, and the like, and can be determined in a routine manner.

In particular embodiments, more than one administration (e.g., two, three, four or more administrations) may be employed to achieve the desired level of dosing over a period of various intervals, e.g., hourly, daily, weekly, monthly, yearly, etc.

In particular embodiments, administration can be local or systemic. For example, delivery of NTSR1-targeted ligand encompasses situations in which a NTSR1-targeted ligand is delivered to a target tissue and the NTSR1-targeted ligand is substantially retained within the target tissue (also referred to as “local distribution” or “local delivery”), and situations in which a NTSR1-targeted ligand is delivered to a target tissue and the NTSR1-targeted ligand is secreted into a patient's circulation system (e.g., serum) and systematically distributed and taken up by other tissues (also referred to as “systemic distribution” or “systemic delivery”). The delivery can also be to airway epithelial cells or any tissue affected by cancer such as epithelial cells from lung, nose, ear, eye, nervous system, and the gastrointestinal and reproductive tract tissues.

Injectables can be prepared in conventional forms, either as liquid solutions or suspensions, solid forms suitable for solution or suspension in liquid prior to injection, or as emulsions. Alternatively, one may administer the NTSR1-targeted ligand of the invention in a local manner.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, cerebrospinal, and inhalation administration, otic administration, ocular administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being detected or treated and on the nature of the particular compound which is being used.

For injection, the carrier will typically be a liquid, such as sterile pyrogen-free water, pyrogen-free phosphate-buffered saline solution, bacteriostatic water, or Cremophor EL[R](BASF, Parsippany, N.J.). For other methods of administration, the carrier can be either solid or liquid.

The NTSR1-targeted ligand can alternatively be formulated for nasal, otic, or ocular administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

### Example 1

Problem to be solved. The high incidence of NTSR1 in prostate cancer makes it a promising target for prostate cancer companion imaging and therapy. However, native NTS has a short half-life in the blood due to quick degradation by endogenous proteases and peptidases, making it unsuitable for imaging/therapy applications. Based on stabilized NTS analogs, several NTSR1 PET agents (labeled with ¹⁸F and ⁶⁴Cu) that demonstrated high contrast in the imaging of NTSR1 positive tumor models have been developed. In a PC3 tumor model, the PET agent ¹⁸F-VS-Cys-NTSₘᵤₜ demonstrated 19.4±5.5 (tumor/muscle), 15.6±4.1 (tumor/liver), and 3.0±0.3 (tumor/kidney) ratios, respectively. However, the agent only demonstrated moderate, 1.3% injected dose per gram (ID/g) tumor uptake and it was cleared out from mice within a few hours. A peptide based NTSR1 agent may be suitable for imaging purposes, but not for therapy applications. An NTSR1 theranostic agent may have both high contrast and prominent and persistent tumor uptake. NTSR1 can be selectively inhibited by SR48692 or its derivative SR142948, a class of nonpeptide antagonist that binds preferentially to NTSR1 and inhibits its downstream signaling pathways.

Solution 1) The discovery of a new NTSR1 ligand SR-CP-05 for targeted imaging and therapy. The introduction of crosslinked polyamines into SR142948A (leading to the new agent, SR-CP-05) could increase the tumor uptake by 10 times compared with peptide based agents. Although the tumor to muscle contrast was reduced somewhat right after injection, the ratio is still >20 at 1 hr post injection. More importantly, the agent stayed within the tumor for up to 48 hr, making it useful for therapeutic applications as well.

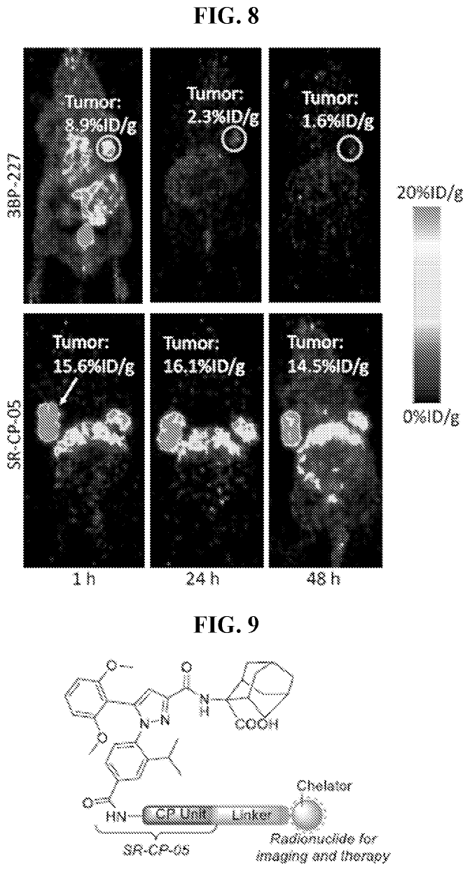

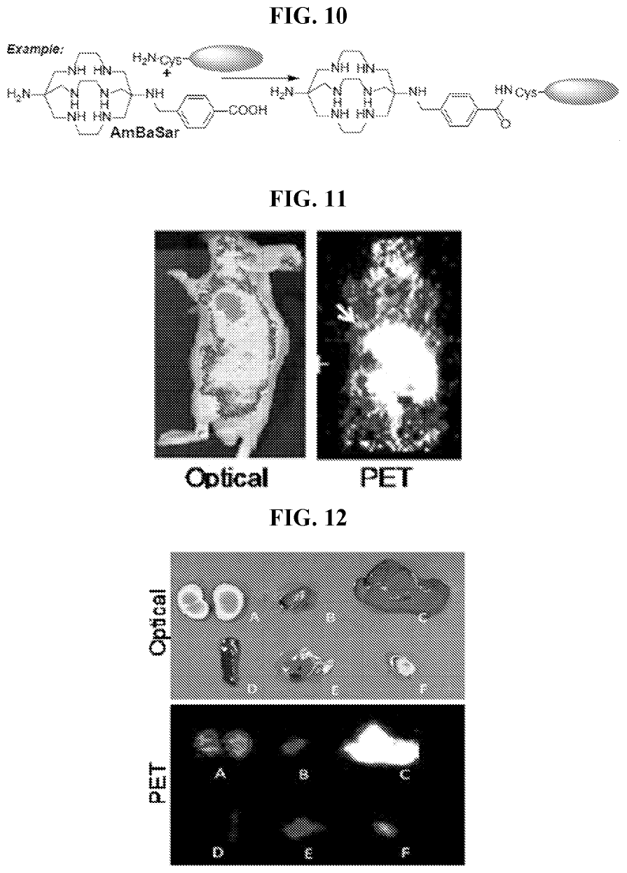

The NTSR1 targeted imaging agent was constructed based on NTS peptide derivatives. Although high tumor-to-background contrast could be obtained, the absolute tumor uptake was moderate with fast clearance from the subject. Moreover, these NTSR1 binding peptides are generally agonists of NTSR1, which may promote tumor progress at high concentration. This may not be a concern for PET imaging due to the limited amount, but it could be a potential risk in treatment. Antagonistic SR142948A based agents were developed, the lead agent being 3BP-227. Side by side comparisons between the lead agent SR-CP-05 and previously reported agents were performed. As shown in FIG. 8, SR-CP-05 showed a 15.5% injected dose per gram (ID/g) tumor uptake (more than 10 time higher than peptide probes, and 75% higher than 3BP-227 (8.9% ID/g)) and high contrast (tumor/muscle >20, compared with tumor/muscle >3 at 1 hr post injection for 3BP-227). Importantly, SR-CP-05 maintained >15% ID/g tumor uptake at 48 hr post injection. In contrast, the tumor uptake of 3BP-227 decreased to 2.3% ID/g at 24 hr post injection. The unique distribution profile of SR-CP-05 can provide an imaging and radionuclide-based therapy agent targeting NTSR1.

2) The radiolabeling methods are innovative. Sarcophagin (Sar) cage was used for stable ⁶⁴Cu chelation. Sar cage was demonstrated to significantly increase tumor to background contrast potentially due to its cage structure and positive charge. These positively charged chelators match the positive charge of the polyamine in SR-CP-05, potentially reducing the impact of the chelator towards the binding ligand.

These novel agents may be used for patient screening, radionuclide-based therapy, and treatment monitoring, all of which are anticipated to impact the care of patients with advanced prostate cancer. In summary, SR-CP-05 ligand may be an innovative product for prostate cancer patient management through NTSR1 targeted imaging and therapy.

Although this example was focused on prostate cancer, it should be noted that NTSR1 is also upregulated in numerous other solid tumors, including lung, head and neck, colorectal, and breast cancers. Therefore, the agents may be expanded to other tumor types.

NTSR1, PSMA, and GRPR expression in prostate cancer patient tissues has been evaluated. It is important to determine the NTSR1 expression profile at various states of prostate cancer in patient samples. NTSR1 expression in both normal prostate and localized prostate cancer was evaluated. Among 97 prostate cancer patient samples that were evaluated, 94 samples showed high NTSR1 expression and 3 samples showed moderate NTSR1 expression (FIG. 2B), whereas normal prostate samples showed low to negative NTSR1 expression (FIG. 2A). In addition to NTSR1, the same prostate patient tissues were stained for PSMA and GRPR expression. Among 75 PSMA staining samples 14 samples (18.7%) showed moderate to low PSMA expression. Among 72 GRPR staining samples, 14 samples (19.4%) showed moderate to negative GRPR expression. Significantly, for all the samples when PSMA and GRPR expression were moderate to negative, NTSR1 was highly expressed. A representative example is shown in Table 1 (NTSR1 high, PMSA low, and GRPR moderate). These preliminary staining studies clearly demonstrated NTSR1 could complement PSMA/GRPR for more precise prostate cancer management.

TABLE 1

| | | | |
|---|---|---|---|
| Expression | NTSR1 | PSMA | GRPR |

| | | | | | | |
|---|---|---|---|---|---|---|
| High | 94 | (96.9%) | 61 | (81.3%) | 58 | (80.6%) |
| Moderate | 3 | (3.1%) | 11 | (14.7%) | 5 | (6.9%) |

| | | | | | |
|---|---|---|---|---|---|
| Low | 0 | 3 | (4%) | 7 | (9.7%) |

| | | | | |
|---|---|---|---|---|
| Negative | 0 | 0 | 2 | (2.8%) |

| | | | |
|---|---|---|---|
| Total # | 97 | 75 | 72 |

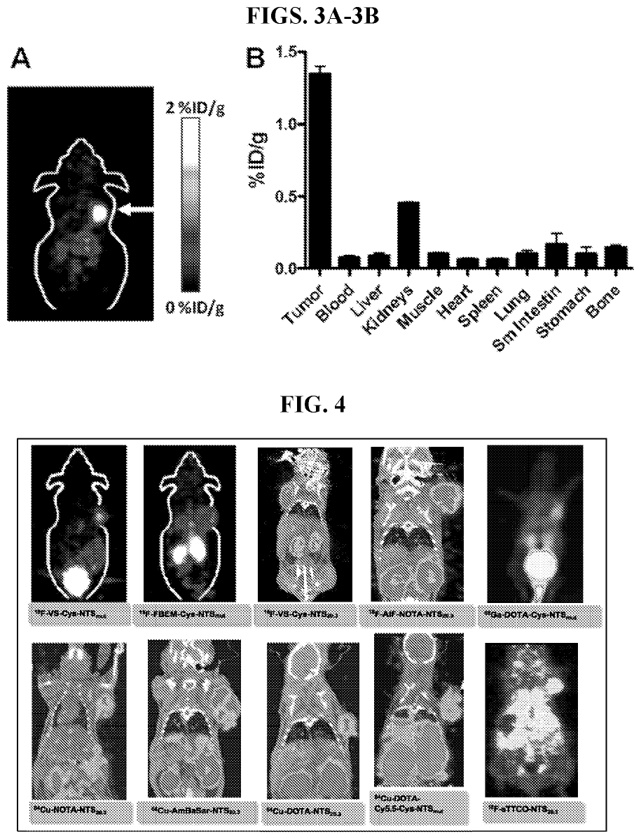

Stabilized NTS peptide derivatives and constructed NTSR1 PET agents have been developed. A stabilized NTS analog with a Cys for ¹⁸F labeling (Cys-NTSₘᵤₜ: Cys-pipGly-Pro-pipAmGly-Arg-Pro-Tyr-tBuGly-Leu-OH) was designed. A representative example is shown in FIG. 3. ¹⁸F-DEG-VS-NTS demonstrated good tumor uptake (1.3±0.1% ID/g) and low background in a PC3 tumor model. Biodistribution studies were performed at 3 hr post injection, showing the tumor to muscle, liver, and kidney ratios at 19.4±5.5, 15.6±4.1, and 3.0±0.3, respectively. Cold NTS peptide successfully blocked the tumor uptake of ¹⁸F-DEG-VS-NTS in PC3 tumor, demonstrating the receptor specificity of this imaging agent. Clearly, ¹⁸F-DEG-VS-NT showed low background in major tissues/organs, including blood, muscle, liver and kidneys. Despite the high contrast, its tumor retention is low.

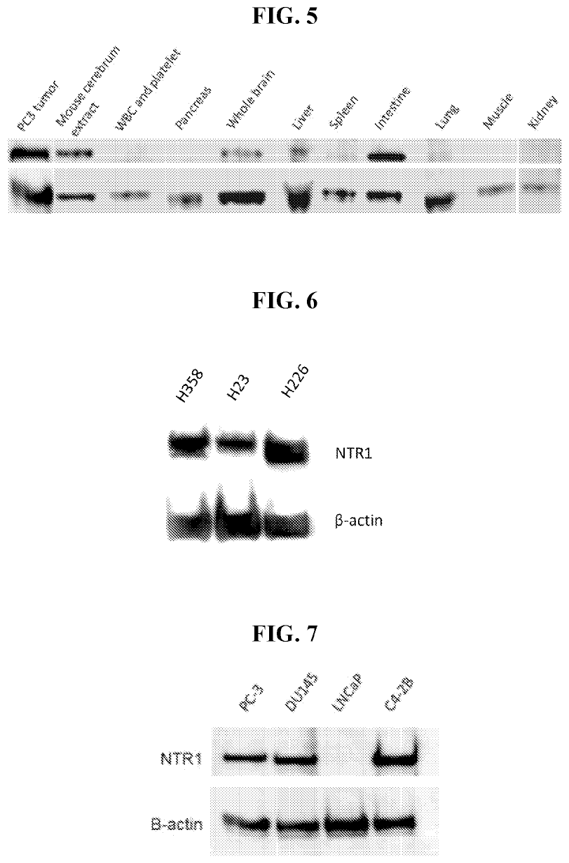

Most normal tissues were demonstrated to have low NTSR1 expression. There are concerns that the presence of NTSR1 in normal tissues may lead to high background uptake and low contrast. Therefore, western blot analysis was performed to evaluate relative expression of NTSR1 in tumor and normal organs. Mouse cerebrum was used as a positive control. As shown in FIG. 5, in most organs (including spleen, liver, lung, pancreas, muscle, white blood cell and platelets), NTSR1 protein was either very low or not detected. NTSR1 was mainly observed in intestine, cerebrum, and PC-3 tumor. As shown in FIGS. 3A-3B, the brain has minimal NTSR1 targeted tracer uptake as the agent would not cross the blood brain barrier (BBB). Although small intestine showed high NTSR1 expression, its tracer uptake is only ⅙ of that in PC-3 tumor, based on the biodistribution experiments (FIGS. 3A-3B). Therefore, radiation to this sensitive organ should be less of a concern. Overall, background uptake should not be a major concern based on this preliminary data.

Various advanced prostate cancer cell lines have high NTSR1 expression. NTS and NTSR1 are induced in advanced prostate cancer as an alternative growth pathway. In order to validate NTSR1 would be a valid target for advanced prostate cancer, western blot analysis was performed on four prostate cancer cell lines: LNCaP (derived from lymph node metastasis), C4-2B (bone metastatic subline generated from LNCaP), DU145 (moderate metastatic potential), and PC-3 (high metastatic potential, PSMA negative). As shown in FIG. 7, LNCaP cells showed minimal NTSR1 as the parent cell, but high NTSR1 expression as it becomes bone metastatic subline C4-2B. Prostate cancer cell PC3 showed prominent NTSR1 expression but low PSMA expression. Clearly, NTSR1 target imaging and therapy could be an excellent complement to current prostate cancer management.

SR-CP-05 was discovered to be well suited for therapy applications. Although NT analogs NTSₘᵤₜ and the NTS_{20.3} peptides already demonstrated promising tumor imaging results, the absolute tumor uptake value is only ˜1.5% ID/g, which was quickly cleared out at 3-4 hr post injection. Clearly, the fast clearance may be acceptable from an imaging point of view, but these ligands are not suited for therapeutic applications. After extensive searching and modifying various NTSR1 ligands, it was discovered that after introducing crosslinked polyamines to SR142948A, the resulting agent SR-CP-05 showed a 15.6% ID/g tumor uptake (FIG. 8), more than 10 time higher than peptide probes, and 75% higher than 3BP-227 (an agent based on SR142948A) in the side-by-side comparison study. Importantly, high contrast (tumor/background >20) was obtained at 1 hr post injection and the washout was minimal even after 48 hr post injection. In contrast, peptide-based agents showed significant washout even at 4 hr post injection; and 3BP-227 demonstrated much lower tumor to background ratio (only ˜3-4) at early time points (FIG. 8) and only ⅓ of tumor uptake was maintained at 24 hr post injection in a side-by-side comparison. Clearly, SR-CP-05 represents a highly promising ligand for both imaging and therapy applications. In summary, NTSR1 was demonstrated as a valid target for prostate cancer management and used as a lead agent, SR-CP-05 showed both high and persistent tumor uptake.

Constructing NTSR1-targeting ligands based on SR-CP-05. Although not wishing to be bound by theory regarding the greatly improved tumor uptake and retention of SR-CP-05, it is believed that the positive charge of the crosslinked polyamine increased the interaction of SR-CP-05 with the cell membrane, enhancing the chance of binding with NTSR1 and retention. The radiolabeled NTSR1 targeted radiopharmaceutical can be divided into three parts: the NTSR1 targeting ligand, bifunctional linker, and the radionuclide component (FIG. 9).

### Example 2

Problem to be solved. NTSR1 is a target for lung cancer companion imaging and therapy. From an imaging point of view, the development of PET agents to obtain NTSR1 cell expression profiles or “fingerprints” of individual tumors could be used for patient screening and treatment monitoring of non-small cell lung cancer (NSCLC) patients. An NTSR1 targeted PET agent may allow the identification of patients with poor prognosis within a determined stage, which may lead to corresponding more suitable postoperative treatments (personalized therapy). A PET probe needs to have a low background in the abdomen area (including both the liver and kidneys) in order to efficiently detect the small lesions around that area. A number of radiolabeled NTS peptide analogues have been developed as a valuable tool for both imaging and therapy of NTSR1-positive tumors. Several NTSR1 PET agents (including ¹⁸F agents labeled through tetrazine-trans-cyclooctene (TTCO) ligation) have been developed that demonstrated high contrast (moderate tumor uptake with minimal accumulation in the upper body and most of the abdomen area including the kidney and liver). Details are shown in the results (FIGS. 3A-3B, 4). Although encouraging results have been obtained in these imaging studies, several challenges need to be addressed in order to develop these probes into clinically useful probes. First, NTS is a tridecapeptide that has a short half-life in the blood due to quick degradation by endogenous peptidases. Changes have been introduced to help stabilize the bonds between Arg8-Arg9, Pro10-Tyr11, and Tyr11-Ile12 and provide metabolic stability. For example, NT-20.3 peptide was demonstrated to be an NTSR1 ligand with good stability for diagnostic or therapeutic purposes. However, the resulting agents were still cleared out from mice within a few hours.

NTSR1 may be a target for lung cancer progression. Therefore, NTSR1 targeted imaging and therapies may become important imaging/treatment strategies for managing lung cancer. A NTSR1 targeted imaging agent was constructed based on NTS peptide derivatives. Although high tumor to background contrast could be obtained, the absolute tumor uptake was moderate with a fast clearance rate from the subject. Moreover, these NTSR1 binding peptides are generally agonists of NTSR1, which may promote tumor progress at high concentration. This may not be a concern for PET imaging due to the limited amount that may be administered, but it did increase the risk potential. A SR142948A-based agent was developed, the lead agent being 3BP-227. There is a need to develop NTSR1 targeted imaging and therapy agents with high and persistent tumor uptake with relatively low background.

Solution 1) The discovery of a new NTSR1 ligand SR-CP-05 for targeted imaging and therapy. The introduction of crosslinked polyamines into SR142948A (leading to the new agent, SR-CP-05) could increase the tumor uptake by 10 times compared with peptide based agents. Although the tumor to muscle contrast was reduced somewhat right after injection, the ratio is still >20 at 1 hr post injection. More importantly, the agent stayed within the tumor for up to 48 hr, making it useful for therapeutic applications as well. Side by side comparisons were made between SR-CP-05 and previously reported agents. As shown in FIG. 8, The tumor uptake of SR-CP-05 showed a 15.5% ID/g tumor uptake, more than 10 times higher than peptide probes, and 75% higher than 3BP-227. Importantly, high contrast (tumor/muscle >20) was also obtained for SR-CP-05 at 1 hr post injection and the washout is minimal after 48 hr post injection (compared with tumor/muscle >3 at 1 hr post injection for 3BP-227). Moreover, both peptide-based agents and 3BP-227 showed significant washout at 24 hr post injection. The uptake of 3BP-227 also decreased to 2.3% ID/g at 24 hr post injection from 8.9% ID/g at 1 hr post injection; while the tumor uptake of SR-CP-05 was maintained >15% ID/g for both time points. The unique distribution profile of SR-CP-05 provides an imaging and radionuclide-based therapy targeting NTSR1.

These novel agents may be used for lung cancer prognosis, treatment monitoring, and radionuclide-based therapy, all of which may impact the care of patients with NSCLC. NTSR1 has been found to be upregulated in numerous other solid tumors. Therefore, agents described herein can be expanded to other tumor types including prostate, head and neck, colorectal, and breast cancers.

Robust ¹⁸F-labeling methods for facile preparation of ¹⁸F-NTS agents based on NT peptide derivatives were developed. As one of the commonly used PET radioisotopes, ¹⁸F can be easily produced in high quantities on a medical cyclotron with an ideal half-life of 110 min for imaging applications. The half-life of ¹⁸F is sufficient to allow synthesis, transportation, and imaging procedures to be extended over several hours, while the patient is subjected to a limited amount of radiation exposure. A platform technology based on vinyl sulfone and TTCO ligation was developed, both of which are highly efficient for ¹⁸F PET probe construction. After establishing efficient ¹⁸F labeling methods, a stabilized NTS analog was designed with a Cys for ¹⁸F labeling (Cys-NTSₘᵤₜ: Cys-pipGly-Pro-pipAmGly-Arg-Pro-Tyr-tBuGly-Leu-OH). Both ¹⁸F-VS and TTCO ligation have been used to construct ¹⁸F labeled PET agents targeting NTSR1. A representative example is shown in FIGS. 3A-3B. ¹⁸F-DEG-VS-NTS demonstrated good tumor uptake (1.3±0.1% ID/g) and low background in a NTSR1 positive PC3 tumor model. A biodistribution study was performed at 3 hr post injection, which showed the tumor to muscle, liver, and kidney ratios were 19.4±5.5, 15.6±4.1, and 3.0±0.3, respectively. In the presence of cold NTS peptide, the uptake of ¹⁸F-DEG-VS-NTS in tumor was efficiently blocked, clearly demonstrating the receptor specificity of this imaging agent. Clearly, ¹⁸F-DEG-VS-NT showed low background in major tissues/organs, including blood, muscle, liver, and kidneys. The very low background in normal tissues allowed the detection of small tumor metastasis (especially around the liver area) by PET imaging.

PET agents targeting NTSR1 were developed. In addition to ¹⁸F-VS labeled agents targeting NTSR1, other labeling methods and radioisotopes to label NTS peptide analogs were explored. Various radiolabeled NTS peptides as pilot compounds were synthesized and screened for their imaging properties and target specificity. Selected chemical structures are shown below and representative NTSR1 targeted imaging are shown in FIG. 4. Overall, these peptide-based imaging agents demonstrated high tumor to background contrast and fast clearance from background including kidney. The limitation would be moderate tumor uptake and the fast clearance profile, which would make them unsuitable for radionuclide-based therapy applications.

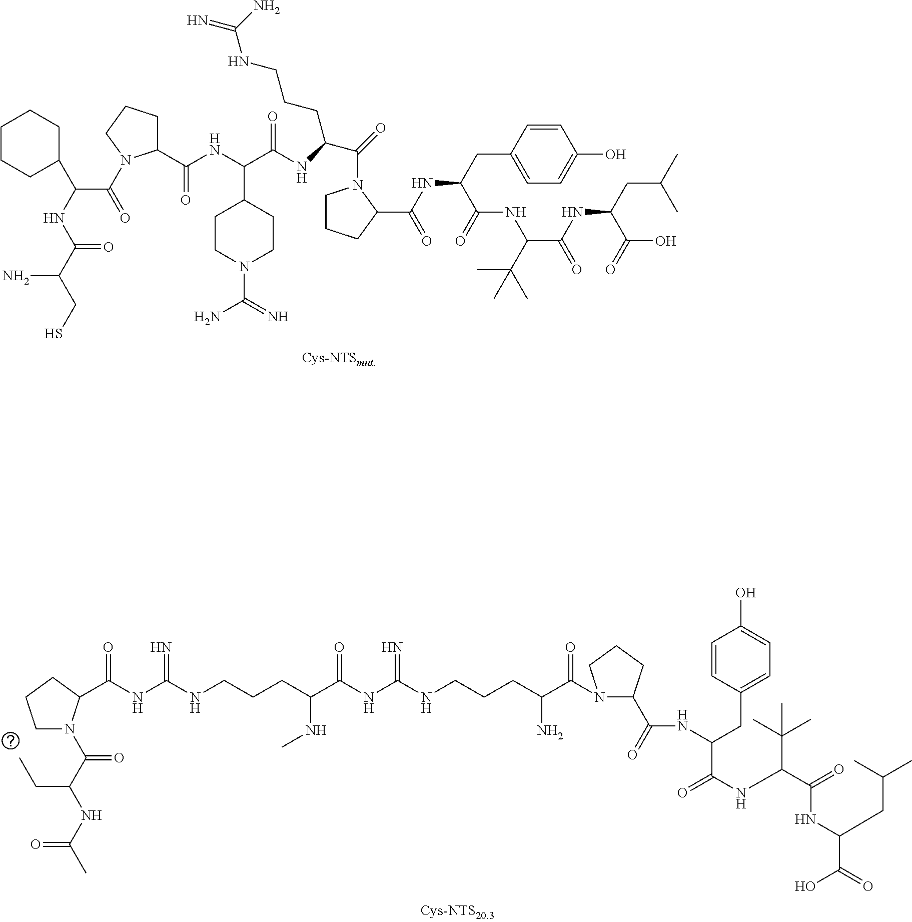

Most normal tissues were demonstrated to have low NTSR1 expression, eliminating potential concerns on target selectivity towards tumor. There are concerns that the presence of NTSR1 in normal tissues may lead to high background uptake and low contrast. Therefore, western blot analysis was performed to evaluate relative expression of NTSR1 in tumor and normal organs (FIG. 5). Mouse cerebrum was used as positive control. As shown in FIGS. 3A-3B, most organs (including spleen, liver, lung, pancreas, muscle, white blood cell and platelets), NTSR1 protein was either very low or not detected. NTSR1 was mainly observed in intestine, cerebrum, and NTSR1 positive PC-3 tumor. As shown in FIGS. 3A-3B and 4, the brain has minimal NTSR1 targeted tracer uptake as the compound of the invention would not cross the blood brain barrier. Although small intestine demonstrated high NTSR1 expression, its tracer uptake is only ⅙ of that in NTSR1 positive tumor based on the biodistribution experiments (FIGS. 3A-3B). Therefore, radiation to this sensitive organ should be less of a concern. Overall, the peptide-based lead compound demonstrated very clean background in the upper body and most of the abdomen area, clearly justifying its usage to detect NTSR1 positive lung cancer and metastasis in those areas. Background uptake was not a major concern based on this data (FIGS. 3A-3B and 5).

Various lung cancer cell lines have high NTSR1 expression. NTS and NTSR1 may play key roles in a significant portion of lung cancer patients. H1299, H1975, H23, H226, and H460 were all reported to be NTSR1 positive. H1299 was confirmed to be a lung cancer cell line that has high NTSR expression. A western blot confirmed H23 and H226 cell lines have high NTSR1 expression as shown in FIG. 6. Clearly, NTSR1 targeted imaging and therapy may be an excellent complement to current lung cancer management.

SR-CP-05 was discovered to be well suited for both imaging and therapy applications. Although NT analogs (NTSₘᵤₜ and the NTS20.3 peptides) already demonstrated promising tumor imaging results, the absolute tumor uptake value is only ˜1.5% ID/g, which was quickly cleared out at 3-4 hr post injection. Clearly, the fast clearance may be acceptable from an imaging point of view, but these ligands are not suited for therapeutic applications. It was discovered that modifying various NTSR1 ligands, such as introducing crosslinked polyamines to SR142948A, the resulting agent SR-CP-05 showed a 15.6% ID/g tumor uptake (FIG. 8), which was more than 10 time higher than peptide probes, and 75% higher than 3BP-227 (a previously reported agent based on SR142948A) in the side by side comparison study. Importantly, high contrast (tumor/background >20) was obtained at 1 hr post injection and the washout was minimal even after 48 hr post injection. In contrast, the peptide-based agent showed significant washout even at 4 hr post injection; and 3BP-227 demonstrated much lower tumor to background ratio (only ˜3-4) at early time points (FIG. 8) and only ⅓ of tumor uptake was maintained at 24 hr post injection in a side by side comparison. Clearly, SR-CP-05 represents a very promising ligand for both imaging and therapy applications. The relatively high contrast at early time points also makes it possible to develop ¹⁸F labeled agents for PET imaging based on SR-CP-05.

Two types of NTSR1 targeted agents were developed that either demonstrated high contrast (peptide based agent with moderate tumor uptake but high contrast: minimal accumulation in the upper body and most of the abdomen area including the kidney and liver), or showed high and persistent tumor uptake (SR-CP-05-based agent).

Developing NTRS1-targeted ligands based on SR-CP-05. Although not wishing to be bound by theory relating to the greatly improved tumor uptake and retention of SR-CP-05, it is believed that the positive charge of the crosslinked polyamine increased the interaction of SR-CP-05 with cell membrane, which as a result enhanced the chance of binding with NTSR1 and retention. A new radiolabeled NTSR1 targeted radiopharmaceutical can be divided into three parts: the NTSR1 targeting ligand, bifunctional linker, and the radionuclide component (FIG. 9).

Sar was demonstrated to be a superior chelator for Cu labeling. Copper-64 (t_{1/2}=12.7 hr) decays by β+ (20%) and β− emission (37%), as well as electron capture (43%), making it well suited for radiolabeling proteins, antibodies and peptides, for both PET imaging (β+) and therapy (β+ and β−). Limitations to the chelating agents currently used with ⁶⁴Cu include significant loss of ⁶⁴Cu from the conjugate, leading to high uptake in the liver. In order to overcome this limitation, a new class of bifunctional chelators (BFCs) based on the hexaazamacrobicyclic sarcophagine cage SarAr (1-N-(4-aminobenzyl)-3,6,10,13,16,19-hexaazabicyclo[6.6.6]-eicosane-1,8-diamine) was synthesized. These ligands coordinate metal ions such as Cu²⁺ within the multiple macrocyclic rings comprising the Sar cage structure, yielding extraordinarily stable complexes that are inert to dissociation of the metal ion. The functionalization approach of Sar cage was improved through a direct alkylation (SN2) reaction. The use of Sar cage for ⁶⁴Cu labeling was demonstrated to be superior. A multimodality probe (shown below) was constructed and demonstrated that DOTA would give misleading information due to its stability problem.

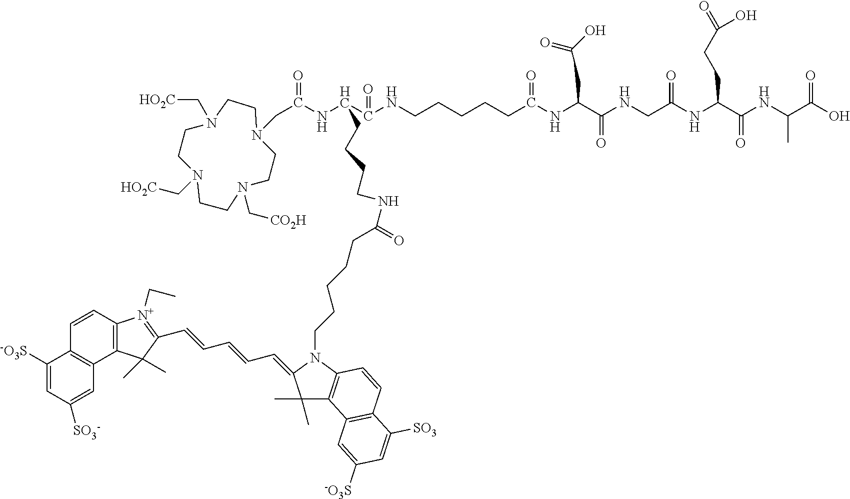

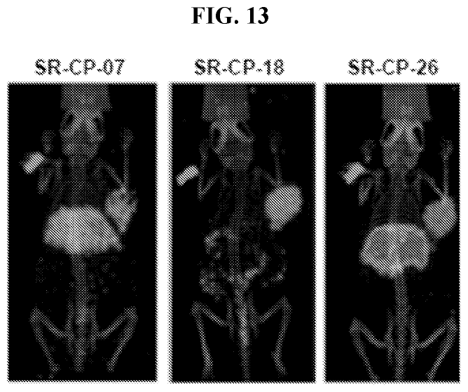

As shown in FIGS. 11 and 12, both PET and optical images demonstrated tumor uptake. However, the kidneys have much higher tracer uptake compared with liver, while the pattern was reversed on PET imaging (Cu-64 falls off DOTA and leads to high liver uptake). This research clearly indicates that Sar may be a preferred ligand for Cu chelation in in vivo applications.

Multifunctionalized Sar cage was demonstrated to hold great potential to efficiently construct PET/optical dual-modality probes. It also clearly indicates that both Sar cage and Cu would not significantly quench the optical signal from the fluorescence dye. This combination of PET imaging with fluorescence may allow fluorescence guided surgery, based on the PET/FL constructs made from the hetero-functionalized Sar cage.

The SR-CP-05 distribution profile was demonstrated in vivo to be suitable for both imaging and therapy applications. However, its liver uptake is still relatively high. Changing the PK linker, SR-CP-18 (NT-1PA-CB) (shown below) mainly showed tumor accumulation at 24 hr time point (high contrast) (FIG. 13), but the tumor uptake is less than half of SR-CP-05.

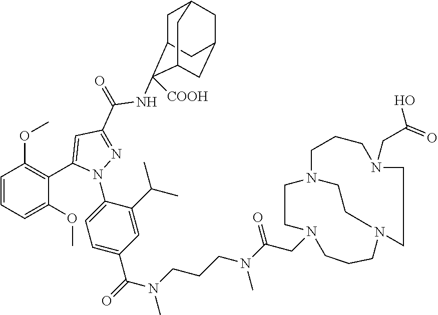 [SMILES: COc1cccc(OC)c1-c1cc(C(=O)NC2(C(=O)O)C3CC4CC(C3)CC2C4)nn1-c1ccc(C(=O)N(C)CCCN(C)C(=O)CN2CCCN3CCN(CCCN(CC(=O)O)CC3)CC2)cc1C(C)C]

Clearly, the distribution of SR-CP-05 derivatives could be tuned by linkers. ⁶⁴Cu/⁶⁷Cu is a true-theranotic radionuclide pair. Unlike ⁶⁸Ga (t_{1/2}=68 min, imaging) and ¹⁷⁷Lu (t_{1/2}=6.7 days, therapy) pair, the half-life between ⁶⁴Cu (t_{1/2}=12.7 h, imaging) and ⁶⁷Cu (t_{1/2}=61.8 h, therapy) are better matched between ⁶⁴Cu and ⁶⁷Cu. Moreover, because both of them are Cu²⁻ ions, the discrepancy caused by using different metals is eliminated.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A neurotensin receptor (NTSR1)-specific ligand suitable for radiolabeling, the NTSR1-specific ligand comprising:
a neurotensin ligand;
a linker; and
a chelator for binding a radioisotope.

2. The NTSR1-specific ligand of claim 1, further comprising a radioisotope.

3. The NTSR1-specific ligand of claim 2, wherein the radioisotope is selected from ⁶⁴Cu, ⁶⁷Cu, ⁶⁸Ga, ¹⁷⁷Lu, ¹⁸F, ⁹⁰Y, ²²⁵Ac, ²²⁷Th, ²²³Ra, ²¹³Bi, ²¹¹At, ²¹²Pb, ²¹²Bi, ²³⁰U, ²²⁶Th, and ¹⁴⁹Tb.

4. The NTSR1-specific ligand of claim 2, having a radio purity of at least 95% (e.g., 95%, 96%, 97%, 98%, 99%, or higher).

5. The NTSR1-specific ligand of claim 1, wherein the chelator is NOTA, DOTA, Cross Bridge-Cyclam, Cross Bridge-TE2A, DF chelators, DiAm-Sarcage, or derivatives thereof.

6. The NTSR1-specific ligand of claim 1, wherein the linker is NOTA, DOTA, Cross Bridge-Cyclam, Cross Bridge-TE2A, Sarcage, and derivatives thereof, polyamine, urea, polyethylene glycol, di-vinyl sulfone, di-halogenated alkyl, phenylene isothiocyanate, p-phenylene diisothiocyanate, 1,4-diazinane, phosphates, and combinations thereof.

7. The NTSR1-specific ligand of claim 1, wherein the neurotensin ligand is SR-CP-05.

8. The NTSR1-specific ligand of claim 1, comprising formula I: 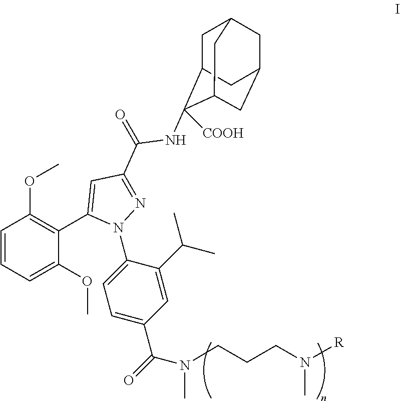
wherein n is 1-3; and
R is a chelator.

9. The NTSR1-specific ligand of claim 8, wherein the chelator is NOTA, DOTA, Cross Bridge-Cyclam, Cross Bridge-TE2A, Sarcage, or derivatives thereof.

10. The NTSR1-specific ligand of claim 1, comprising formula II: 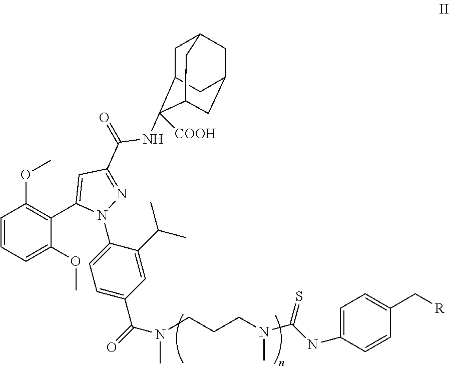
wherein n is 1-3; and
R is a chelator.

11. The NTSR1-specific ligand of claim 10, wherein the chelator is selected from NOTA, DOTA, Cross Bridge-Cyclam, Cross Bridge-TE2A, Sarcage, and derivatives thereof.

12. The NTSR1-specific ligand of claim 1, comprising formula III: 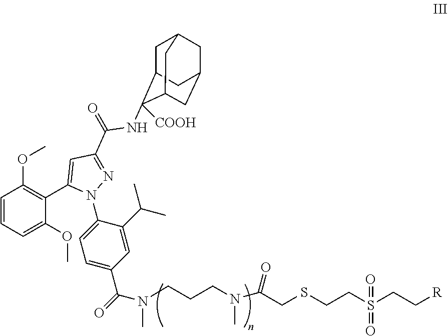
wherein n is 1-3; and
R is a chelator.

13. The NTSR1-specific ligand of claim 12, wherein the chelator is selected from NOTA, DOTA, Cross Bridge-Cyclam, Cross Bridge-TE2A, DiAm-Sarcage, and derivatives thereof.

14. The NTSR1-specific ligand of claim 1, comprising formula IV: 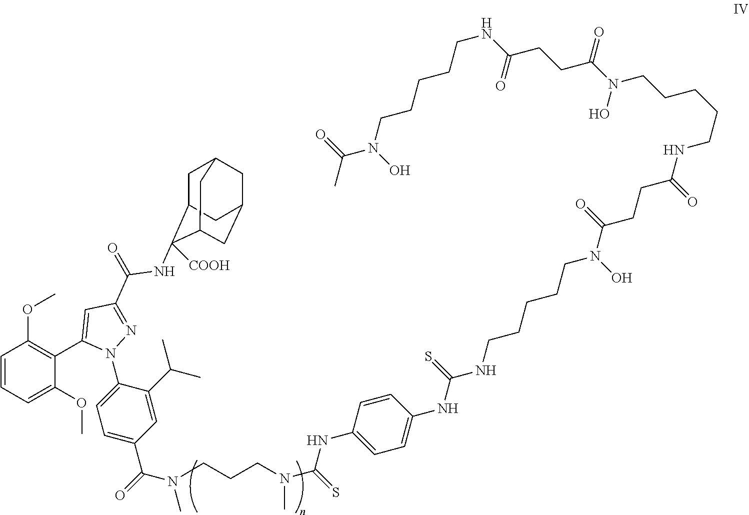
wherein n is 1-3.

15. The NTSR1-specific ligand of claim 1, comprising formula V: 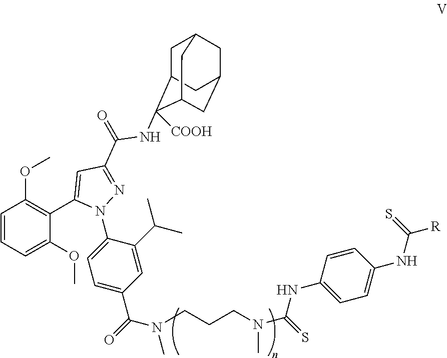
wherein n is 1-3; and
R is a chelator.

16. The NTSR1-specific ligand of claim 15, wherein the chelator is selected from NOTA, DOTA, Cross Bridge-Cyclam, Cross Bridge-TE2A, DiAm-Sarcage, and derivatives thereof.

17. The NTSR1-specific ligand of claim 1, comprising formula VI: 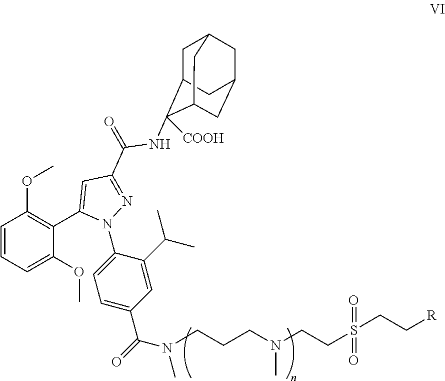
wherein n is 1-3; and
R is a chelator.

18. The NTSR1-specific ligand of claim 17, wherein the chelator is selected from NOTA, DOTA, Cross Bridge-Cyclam, Cross Bridge-TE2A, DiAm-Sarcage, and derivatives thereof.

19. The NTSR1-specific ligand of claim 1, comprising formula VII: 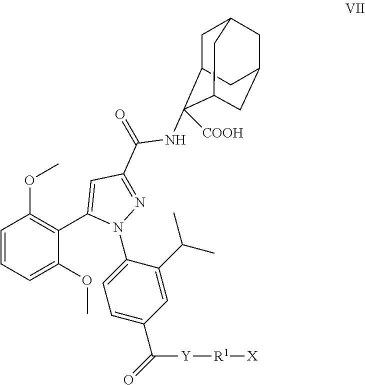
wherein Y is a bond or is selected from PEG, di-vinyl sulfone, di-halogenated alkyl, phenylene isothiocyanate, p-phenylene diisothiocyanate, 1,4-diazinane, and phosphates;
X is H or LR²;
L is selected from PEG, di-vinyl sulfone, di-halogenated alkyls, phenylene isothiocyanate, p-phenylene diisothiocyanate, 1,4-diazinane, and phosphates; and
R¹ and R² are chelators.

20. The NTSR1-specific ligand of claim 19, wherein the chelators are independently selected from NOTA, DOTA, CrossBridge-Cyclam, CrossBridge-TE2A, DiAm-Sarcage, and derivatives thereof.

21. The NTSR1-specific ligand of claim 1, wherein the NTSR1-specific ligand is selected from:

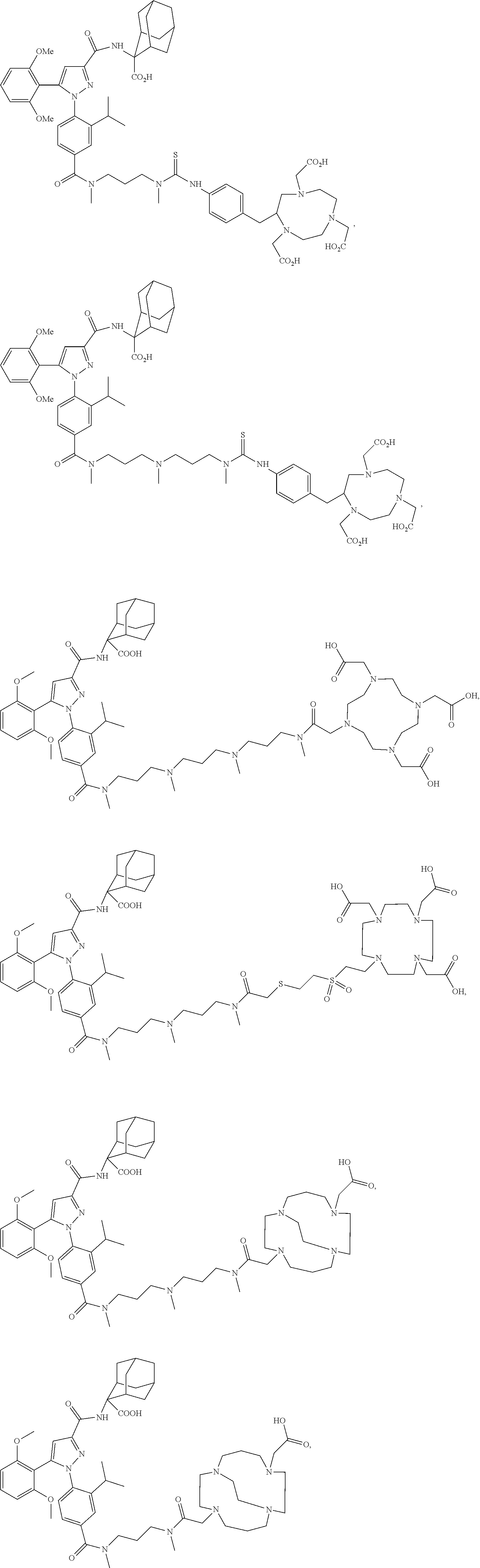

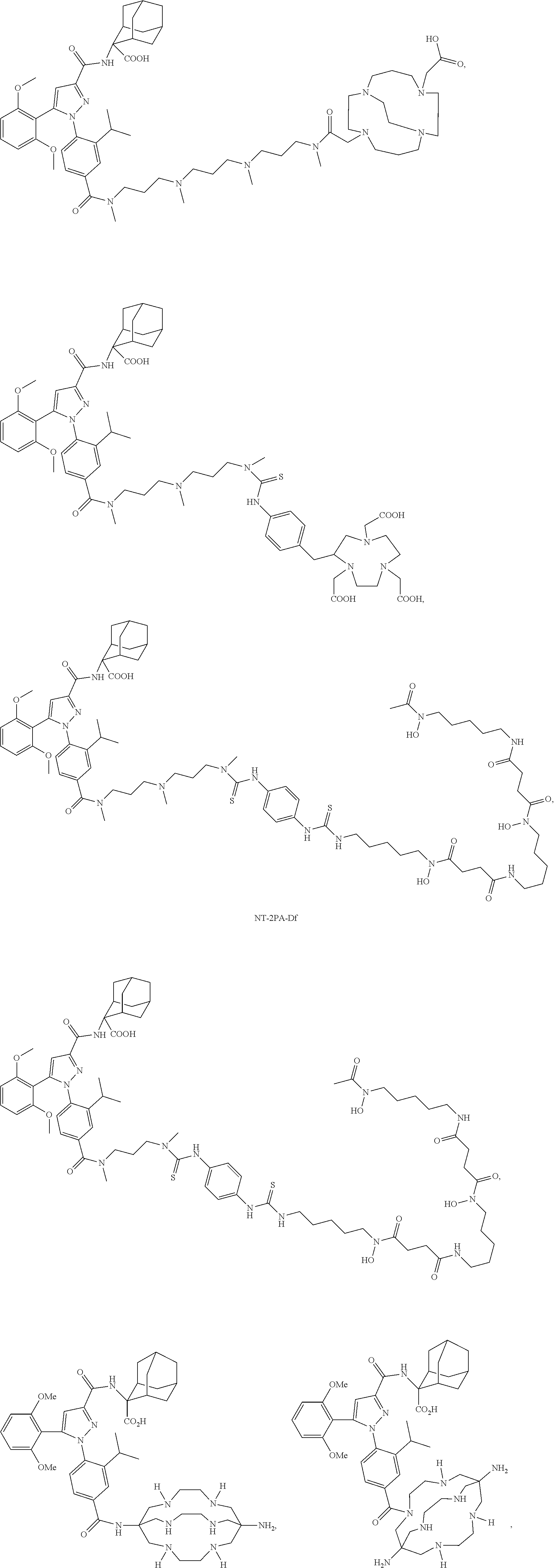

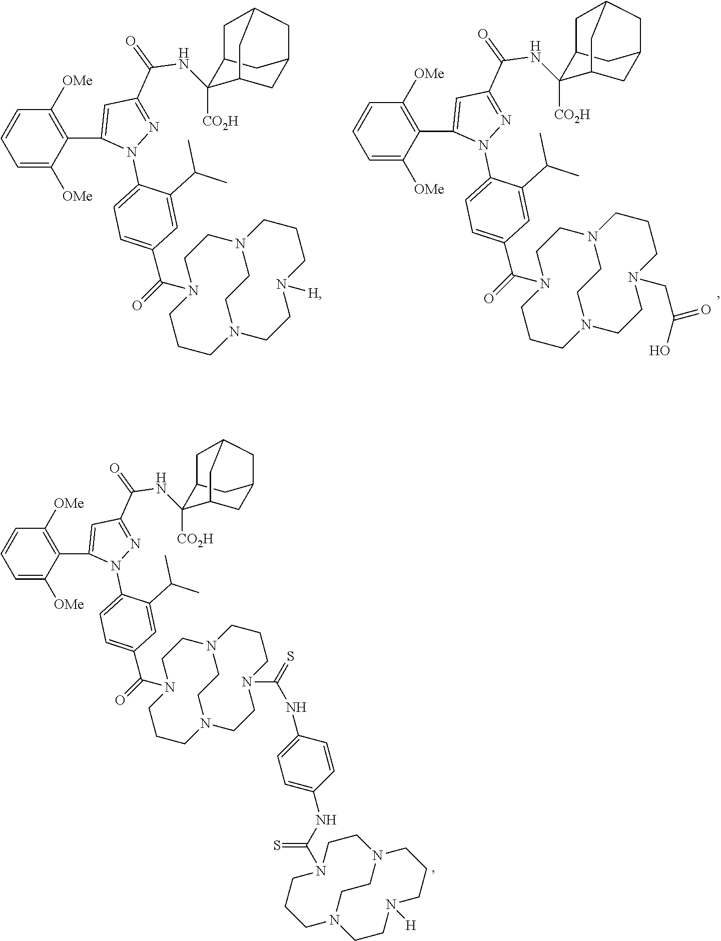

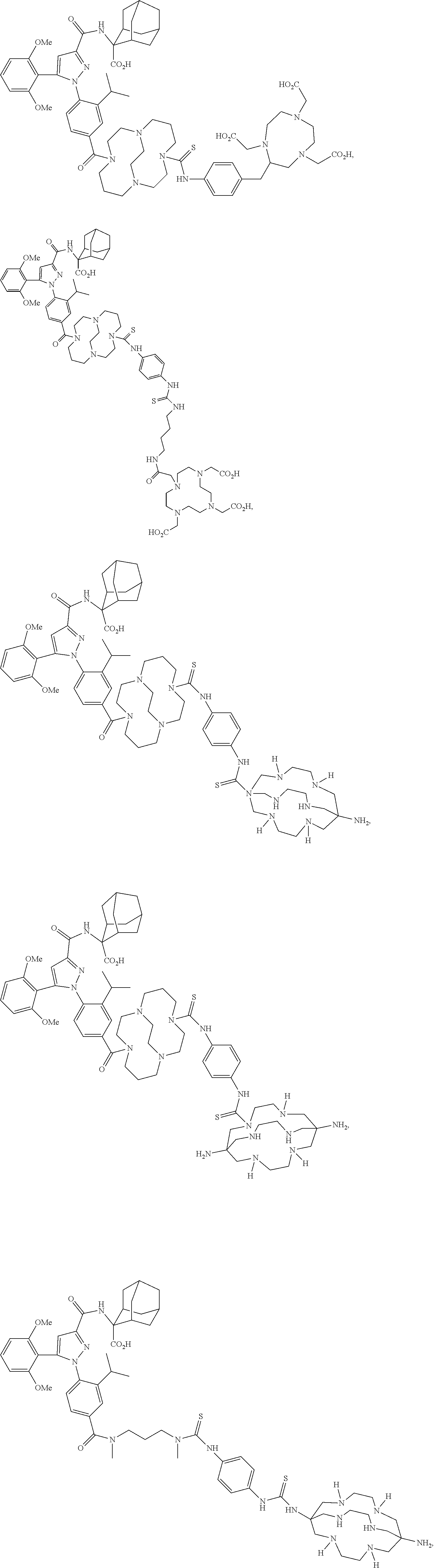

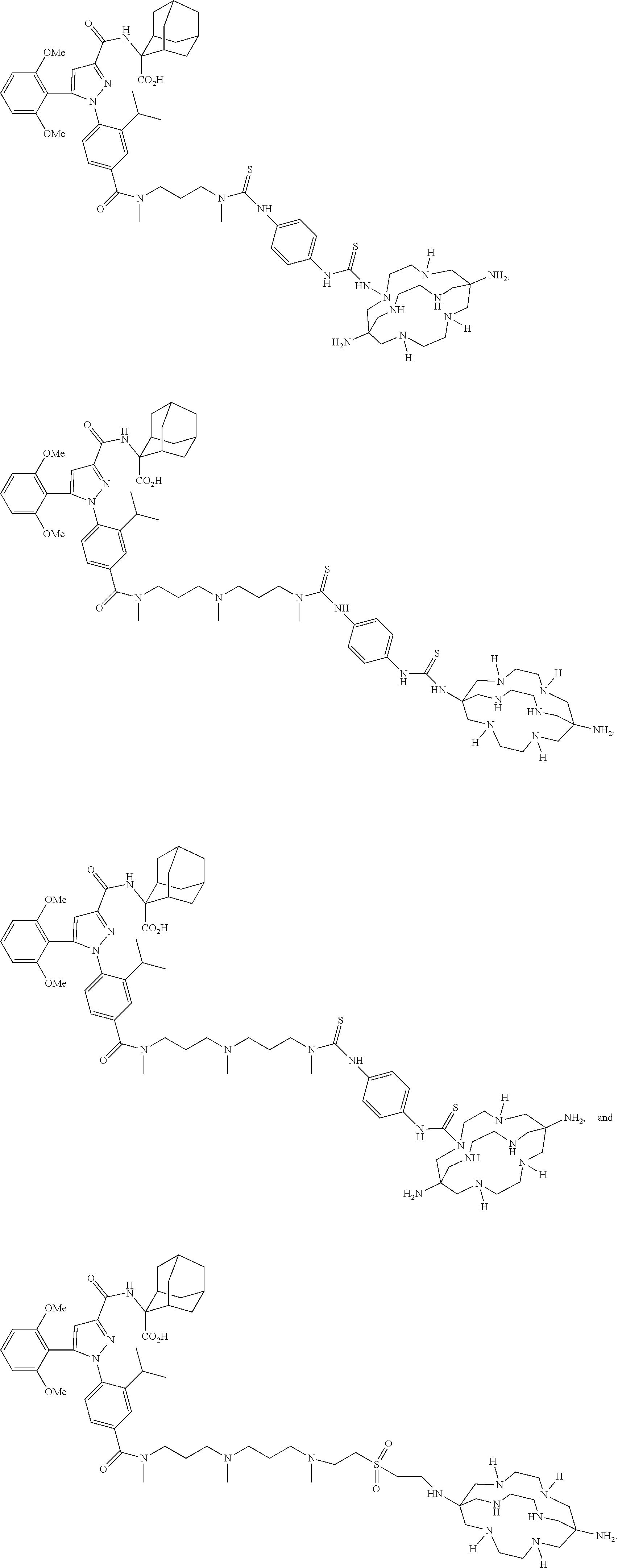

22-51. (canceled)

52. A neurotensin receptor (NTSR1)-specific ligand suitable for radiolabeling, the NTSR1-specific ligand comprising:
a neurotensin ligand;
a linker; and
a chelator for binding a radioisotope, wherein the chelator is selected from the group consisting of CrossBridge-Cyclam, CrossBridge-TE2A, DiAm-Sarcage, and derivatives thereof.

53. The NTSR1-specific ligand of claim 52, wherein the chelator is the DiAm-Sarcage.


SEQUENCE LISTING (1 sequence)

SEQ ID NO: 1 - protein, 9 aa, synthetic construct
  MOD_RES 2: note = pipGly
  MOD_RES 4: note = pipAmGly
  MOD_RES 8: note = tBuGly
CGPGRPYGL